(19) United States
(12) Patent Application Publication
(10) Pub. No.: US 2006/0211044 A1
(43) Pub. Date: Sep. 21, 2006

(54) Translucent solid matrix assay device dor microarray analysis

(76) Inventor: Lawrence R. Green, Tacoma, WA (US)

Correspondence Address: FAEGRE & BENSON LLP;PATENT DOCKETING, 2200 WELLS FARGO CENTER, MINNEAPOLIS, MN, 55402, US

(21) Appl. No.: 10/550,629

(22) PCT Filed: Feb. 17, 2004
(86) PCT No.: PCT/US04/04675

§ 371 Date: Sep. 21, 2005

Related U.S. Application Data
(60) Provisional application No. 60/540,720, filed on Jan. 29, 2004.

(30) Foreign Application Priority Data
Feb. 24, 2003 (US) 10373546
Feb. 24, 2003 (US) 10373408

(51) Int. Cl. G01N 33/53; G06F 19/00; G06K 9/00; C12M 1/34

(52) U.S. Cl. 435/7.1; 435/287.2; 702/19; 382/128

(57) ABSTRACT

The present invention concerns methods and compositions relating to translucent matrix arrays. In certain embodiments, the arrays are reconfigurable. Reconfigurable arrays may be produced using small linker molecules, such as aptamers or affibodies, which preferably bind to an IgG specific portion of antibodies. Such arrays may be used to detect any target that binds selectively or specifically to an IgG. Translucent matrix arrays may utilize cellophane, rayon or a translucent, colloidal form of nitrocellulose to coat the substrate. Other embodiments concern methods of data analysis and apparatus for analyte detection. Certain embodiments concern a total optical assay device (TOAD™), which may be used with GRABBER™ slides and/or FOOTPAD™ microtiter well assay devices.

### RELATED APPLICATIONS

The present application claims the benefit of U.S. patent application Ser. No. 10/373,546 and Ser. No. 10/373,408, each filed on Feb. 24, 2003, and claims the benefit under 35 U.S.C. §119(e) of Provisional U.S. patent application Ser. No. ______, entitled “Methods and Apparatus for Total Optical Assay Device, by Lawrence R. Green, filed on Jan. 29, 2004. The entire text of each above-listed application is incorporated herein by reference.

### BACKGROUND OF THE INVENTION

1. Field of the Invention

The present invention relates to the field of microarray analysis. More particularly, the present invention concerns methods, compositions and apparatus relating to translucent, solid, matrix assay devices for microarray analysis. In certain embodiments of the invention, the microarray may be a reconfigurable microarray. In some embodiments, the methods concern methods of data analysis for optical detection of analytes. Still other embodiments concern apparatus, compositions and methods of use of a total optical assay device (TOAD™), which in preferred embodiments may be used with translucent coated slides or microtiter well plates.

2. Description of Related Art

Detection of trace amounts of various analytes, such as proteins, nucleic acids, lipids, metabolites, toxins, wastes, contaminants, poisons, hormones, pharmaceuticals, drugs, explosives, pathogens, biohazards, hazardous chemicals, viruses, bacteria, cells, diseased cells, etc. is extremely important for many applications in epidemiology, public health, biowarfare, environmental health and safety, forensics, disease detection and treatment and numerous other fields. Bioanalytes in particular pose numerous problems in analyte detection. Many bioanalytes, such as protein variants, may be structurally similar to other related compounds, resulting in cross-reactivity and false positive detection. Other bioanalytes may be relatively unreactive to detection reagents, causing low detection sensitivity and false negative detection.

Numerous methods for detection of proteins and other bioanalytes have been developed, primarily based on some type of immunological assay. Western blots, ELISA assays, sandwich immunoassays, immunoadsorption, etc. all rely upon a binding interaction between an antibody or antibody fragment, such as a monoclonal or polyclonal antibody, and an analyte to be detected. The antibody-analyte complex is then detected, often by addition of a labeled secondary antibody. One format developed for bioanalyte detection has involved the use of microarrays of use for simultaneous analysis of multiple analytes on a single chip format.

Microarray analysis involves the attachment of capture molecules, such as antibodies or oligonucleotides, to a solid matrix. Typically, the array is designed so that capture molecules specific for particular target analytes are attached to identifiable locations on the matrix. After exposure to a sample suspected of containing one or more target analytes, the matrix is analyzed to determine if substances in the sample bind to the capture molecules at one or more locations on the array.

Two-dimensional microarrays have proven useful for a wide range of applications, such as genomic research. Arrays of oligonucleotide probes may be used to determine the match or mismatch for a given sample of DNA or RNA, as in the detection of disease-associated single nucleotide polymorphisms (SNPs). Gene expression-profiling with microarrays containing probes against target gene mRNAs has been used to identify genes that are up- or down-regulated in response to disease, drug treatment, developmental stage and other conditions. Microarrays have also been of use for applications in protein research. However, proteins are more difficult to attach to a solid matrix and far more complex than oligonucleotides. Thus, techniques for use with protein (antibody) microarrays often require modifications compared to the more simple nucleic acid microarrays. (See, e.g., Constans, The Scientist, 16:28, 2002.)

Many clinical diagnostic devices have been built around microarray platforms incorporating an appropriate solid matrix. These often contain capture molecules that have been printed or otherwise permanently affixed to the matrix. One of the problems with such fixed arrays is that they are static. Once an array has been printed, it cannot be changed or adapted to conduct any tests other than the ones that it was originally designed for. A reconfigurable microarray would be very advantageous in allowing flexibility of use.

Existing microarrays face additional problems. For example, the type of solid matrix used may affect the results obtained, depending on the method of analysis and the materials used. Most microarrays are produced using covalent, electrostatic or hydrophobic binding to attach capture probes to the surface of a solid matrix. The capture probes remain attached to the surface during sample analysis. Bound target molecules may be detected in a variety of ways. Most commonly, one or more fluorophore tags are attached to the target molecules or cells that are to be bound by a capture molecule. Once binding is complete the tags may be spectrophotometrically detected. Scanners, CCD cameras or similar detectors may be used to determine the location and signal intensity of fluorescent tags bound to matrix arrays.

The amount of probe material that can be affixed on a matrix surface depends on the composition of the solid matrix. If insufficient amounts of probe are affixed to the matrix, the resulting fluorescent signal will be so weak that it cannot be detected even if the probe captures a tagged target molecule. It is also not sufficient to bind high concentrations of probe molecules to the surface of a solid matrix, if the matrix does not provide sufficient conformational or steric freedom to allow probes to bind to target molecules.

The solid matrix must also preserve the functional activity of the probe. Proteins, such as antibodies, attached to a solid matrix may undergo denaturation over time, rendering antibodies inactive or enzymes dysfunctional. In such cases, the signal strength (and the amount of target protein identified in a sample) may vary by the length of time following matrix array manufacture. Although such time-dependent processes may be compensated for in part by the use of external standard proteins, the denaturation rates for different antibodies or enzymes affixed to the same matrix may not be identical.

Other characteristics of the solid matrix used for 2D arrays may also be important. For example, the opacity of the solid matrix may render it useless for certain kinds of analysis. Opaque materials only allow sample analysis to occur on the same side of the solid matrix as the probe array. This prevents the use of see-through optics that detect light from the opposite side of the matrix. For example, a matrix array may be opposed to a fluidic cube or other fluidic device, with probe molecules attached to the array within a cavity formed by the fluidics cube. Detection of real-time binding of target molecules to the probes would be greatly facilitated if emitted light could be detected from the opposite surface of the array. This is not feasible if the array is opaque to the emitted light. A need exists for a translucent solid matrix that could be used with a variety of optical detection systems. Such translucent matrix materials should also allow for binding of high concentrations of probe molecules, while maintaining probe molecules in an active state.

Microarrays may be used in combination with optical image sensors for detection of analytes. Among possible analytic uses for image sensors is the evaluation and characterization of light interactions between one or more substances on a surface or in a reaction vessel. Light emitted by the object of interest or absorbed by the object when a beam of light passes through it may be detected and quantified.

Most modern light detectors are designed to capture a spectral signal by presenting a two-dimensional array of sensitive photodiodes towards a target. The photodiodes are designed to produce current when exposed to light, and the resulting current may be analyzed in various ways. Modern sensors convert the analog photodiode signal to a digital signal format that may then be stored and processed for later analysis.

Image sensors are often sensitive and responsive, acting to minimize background noise and interference. Image sensors are capable of accurately recording data regarding light striking the photodiode array of the sensor. High-resolution digital pictures may be produced pixel by pixel with an appropriate source of light, an optical system, an image sensor, and a computer. Using such a system, photographic pictures may be obtained in either monochromatic or color formats.

However, a photodiode will produce an analog output signal that correlates with the energy striking the photodiode array only in special circumstances, such as when the target is illuminated by monochromatic light at a particular wavelength. Even though the output signal of a photodiode is essentially linear with respect to the illumination applied to the photodiode, the signal value for a pixel does not generally correlate accurately with the photon flux. This is because the quantum efficiency (QE) for converting the photon flux to a photodiode electrical energy varies with certain factors. In addition, in most cases more than a single wavelength of light will strike a photodiode.

Every photodiode has a certain QE factor that will vary with factors such as wavelength and temperature. Photon flux represents the electromagnetic energy striking the surface of a two-dimensional array, and the QE represents the capability of the photodiode to convert that energy into electrical energy. QE is usually expressed as a percentage of the energy flux, equaling some percentage less than 100 percent. Because QE varies greatly with the wavelength of light illuminating a photodiode, comparisons of a signal at one wavelength to that at another are difficult to interpret unless the QE factors are known for all wavelengths that apply.

Further, most image sensors are designed by manufacturers to produce images that approximate the equivalent of what would be seen on a film or by the human eye. Manufacturers are interested in reproducing “life-like” pictures and colors. Manufacturers may provide access to the raw digital information for every pixel, but image sensors generally process that information before it is available for analysis to better render the “life-like” colors and intensities that represent human visual expectations.

For these reasons, the data produced by an image sensor generally does not directly relate to the photon flux that impinges upon the photodiode array of the sensor. This factor limits the usefulness of image sensors for analytic purposes.

### SUMMARY OF THE INVENTION

The present invention resolves a long-standing need in the art by providing compositions for and methods of production and use of translucent solid 2D matrix arrays. In certain embodiments of the invention, the matrix arrays are microarrays, with binding molecule spots of between 1 μm and 999 μm in size. In particular embodiments, the spots may be between 100 and 500 μm in size. More particularly, the spots may be about 300 μm in size. In specific embodiments, the microarrays may be reconfigurable. Exemplary microarrays of potential use in the claimed methods are disclosed, for example in U.S. patent application Ser. No. 10/035,367, filed Dec. 28, 2001, the entire contents of which are incorporated herein by reference.

In various embodiments of the invention, the solid matrix arrays may exhibit one or more of the following characteristics: [1] inexpensive to produce; [2] long term stability (retain characteristic features over time); [3] ease of manufacture; [4] reproducible target molecule detection and/or quantification between lots; [5] bind high levels of probes; [6] do not interfere with probe functionality (e.g., binding affinity for target molecules); [7] translucent; and [8] do not interfere with probe-target molecule interaction. Such characteristics of the disclosed solid matrix arrays are advantageous compared to previously known solid matrix arrays.

Certain embodiments of the invention concern efficient methods for producing a translucent (optically clear) nitrocellulose matrix supported on a solid substrate. Such translucent matrices are ideally suited for 2D microarray analysis. The translucent nitrocellulose matrix preserves protein (antibody) functionality and exhibits many of the characteristics listed above, such as high binding capacity. The translucent nitrocellulose solid matrix is suitable for genomic and proteomic work and can be used in a variety of diagnostic formats. Because it is translucent, it is suitable for use with see-through optics that detect light from the opposite side of the matrix from the capture molecules. A non-limiting example of a fluidics cube type biosensor of use with the claimed methods and compositions is disclosed in U.S. patent application Ser. No. 09/974,089, filed Oct. 1, 2001, the entire contents of which are incorporated herein by reference.

Other embodiments of the invention concern methods for producing a translucent, optically clear signal using small linker molecules bound to the surface of a solid matrix substrate. Aptamers, affibodies (e.g. U.S. Pat. No. 5,831,012), or other linkers that exhibit a high affinity for the Fc portion of certain antibodies may be used to attach antibodies or antibody fragments to a solid matrix. The use of such linkers overcomes the tendency for the solid matrix to display a lower binding capacity for larger molecules (e.g., antibodies). The methods may be used in combination with optically clear nitrocellulose matrix substrates to create reconfigurable microarrays. Alternatively, the methods may be used with traditional translucent substrates such as glass microscope slides.

Still other embodiments concern methods of data processing and analysis for optical detection systems, for example to use with detection and/or quantification of analyte binding to 2-D matrix arrays.

Certain embodiments of the invention concern methods, compositions and apparatus for a total optical assay device (TOAD™). In particular embodiments, the TOAD™ may be used with nylon slides coated with a layer of translucent nitrocellulose, rayon or cellophane (GRABBER™ slides) or with coated microtiter well plates (FOOTPADS™).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an exemplary 96 Well Microplate Reagent and Footpad™ Format

FIG. 2 illustrates an exemplary Footpad™, showing an array with index spot and positioning in a reservoir with an inverted eppendorf pipet tip.

FIG. 3 illustrates an exemplary method of use of a TOAD™ apparatus with a translucent nitrocellulose-coated slide (GRABBER™).

FIG. 4 illustrates an illustrative example of a Total Optical Assay Device (TOAD™).

FIG. 5 shows an exemplary kit for use with a TOAD™. In this non-limiting example, GRABBER™ slides are included for analyte detection. Alternative embodiments of kits may comprise 96 well microtiter plates

FIG. 6 illustrates an exemplary set of GRABBER™ slides, along with an incubation sleeve with four reaction chambers.

FIG. 7 shows a schematic diagram, illustrating the components of an exemplary TOAD™ apparatus.

FIG. 8 shows a schematic of the upper surface of a TOAD™ apparatus with the casing 810 removed to show the stage 820.

FIG. 9 shows a non-limiting exemplary embodiment of an array of Footpads™ 910 contained in a Footpad™ holder 920.

FIG. 10 shows a side view of an array of Footpads™ 910 in a holder 920.

FIG. 11 shows a bottom view of an array of Footpads™ 910 in a holder 920.

FIG. 12 illustrates an embodiment of an imaging operation.

FIG. 13 is a flowchart illustrating an embodiment of an imaging process.

FIG. 14 is a flowchart illustrating an embodiment of a leveling process.

FIG. 15 illustrates an embodiment of a computer environment.

FIG. 16 is an illustration of a CMOS image sensor;

FIG. 17 is an illustration of a Bayer color filter mosaic array;

FIG. 18 is an illustration of possible filter arrangements for an image sensor;

FIG. 19 is an illustration of microlens operation in an image sensor;

FIG. 20 is a graph of quantum efficiency of an exemplary image sensor,

FIG. 21 is a flow chart illustrating an embodiment of correction of QE factors;

FIG. 22 is a flow chart illustrating an embodiment of calibration for image sensor optimization;

FIG. 23 is a flow chart illustrating an embodiment of image sensor optimization;

FIG. 24 is a block diagram illustrating an exemplary computer that may be utilized in connection with an embodiment of the invention; and

FIG. 25 is an illustration of light scattering detection.

### DESCRIPTION OF ILLUSTRATIVE EMBODIMENTS

Definitions

Terms that are not otherwise defined herein are used in accordance with their plain and ordinary meaning.

As used herein, “a” or “an” may mean one or more than one of an item.

As used herein, the terms “analyte,” “target” and “target analyte” mean any compound, molecule or aggregate of interest for detection. Non-limiting examples of analytes include a protein, peptide, carbohydrate, polysaccharide, glycoprotein, lipid, hormone, growth factor, cytokine, receptor, antigen, allergen, antibody, substrate, metabolite, cofactor, inhibitor, drug, pharmaceutical, nutrient, toxin, poison, biowarfare agent, biohazardous agent, infectious agent, prion, vitamin, waste product, or any other molecule or atom, without limitation as to size. “Analytes” are not limited to single molecules or atoms, but may also comprise complex aggregates, such as a virus, bacterium, Salmonella, Streptococcus, Legionella, E. coli, Giardia, Cryptosporidium, Rickettsia, spore, mold, yeast, algae, amoebae, dinoflagellate, unicellular organism, pathogen or cell. In certain embodiments, cells exhibiting a particular characteristic or disease state, such as a cancer cell, may be target analytes. Virtually any chemical or biological compound, molecule or aggregate could be a target analyte. “Bioanalytes” are “analytes” that are associated with, part of, contained within, incorporated into, metabolites of and/or products of biological systems (e.g., organisms, cells, bacteria, viruses, etc.)

As used herein, “capture molecule” or “probe” refers to a molecule or aggregate that has binding affinity for one or more targets. Within the scope of the present invention virtually any molecule or aggregate that has a binding affinity for some target of interest may be a “probe.” “Probes” include, but are not limited to, polyclonal antibodies, monoclonal antibodies, antibody fragments, FAb fragments, humanized antibodies, single-chain antibodies, chimeric antibodies, affibodies, oligonucleotides, polynucleotides, nucleic acids, aptamers, nucleic acid ligands and any other known ligand that can bind to at least one target molecule. In certain embodiments, the capture molecule is specific for binding to a single target, although in other embodiments the capture molecule may bind to multiple targets that exhibit similar structures or binding domains. In preferred embodiments, the capture molecule is an antibody. Primary antibodies for detection of one or more targets of interest may be provided on a pre-spotted slide or may be selected by the user to load onto a slide or other array.

The terms “detection” and “detecting” are used herein to refer to an assay or procedure that is indicative of the presence of one or more specific analytes in a sample, or that predicts a disease state or a medical or environmental condition associated with the presence of one or more specific analytes in a sample. It will be appreciated by those of skill in the art that all assays exhibit a certain level of false positives and false negatives. Even where a positive result in an assay is not invariably associated with the presence of a target analyte, the result is of use as it indicates the need for more careful monitoring of an individual, a population, or an environmental site. An assay is diagnostic of a disease state or a medical or environmental condition when the assay results show a statistically significant association or correlation with the ultimate manifestation of the disease or condition

In a non-limiting embodiment, “GRABBER™ slides” refer to nylon slides coated with a layer of translucent nitrocellulose, as discussed in more detail below. The nitrocellulose may be applied to a surface with an icon relief. Spotting may be done using a spotting template with the icon in a corner (e.g., the upper left or lower right corner) of the template and the nitrocellulose surface exposed. The invention is not limited to the illustrated preferred embodiments and the skilled artisan will realize that other types of slides, including but not limited to glass or other types of plastic slides, may be used in the practice of the claimed methods and apparatus.

A “spotting template” refers to a tool that may hold slides (e.g., up to 6 slides at a time) when applying probes to the surface of a slide. The template may be used to accurately place spots in the proper field that will be obtained in imaging. In an exemplary embodiment, slides may be provided with diamond marker points. Spotting of probes in the region between the diamond points provides for accurate imaging.

In certain embodiments, a “focus template slide” is a glass slide with a semi-translucent template grid pattern identical to the spotting template. The slide may be used on the stage of the TOAD™ to confirm that the test slide has been properly positioned for imaging. It is also useful in helping the user identify the position of a particular spot in the image.

In some embodiments, “index marking solution” refers to a ready formulated index protein with biotin affixed that when spotted on the surface of a slide, serves as a convenient position marker and positive control. The solution may be used without dilution by spotting onto the surface of the slide a column or row of spots in the same manner as is used in affixing primary probe antibodies.

An “incubation sleeve” may refer to a sleeve with a sticky surface that is applied to a nitrocellulose surface, forming an incubation chamber. In certain embodiments, there are two openings on a diagonal in the window that may be used to fill and or empty the chamber during incubation. The sleeve may be affixed to a slide after the 2D array is formed, but before blocking, and may remain in place throughout the test, even during the imaging procedure. Just before imaging, after the luminol test solution is placed in the sleeve incubator, the thin surface outer window sheet may be peeled away to present a totally clear window during imaging.

“TOAD™ bioilluminator” refers to an imaging apparatus, equipped with a stage to accommodate a prepared slide, set of microtiter wells or other device to be optically imaged. The slide or other device may be placed on the stage so that a sleeve incubator (face up) is positioned accurately in the field for imaging. This may be confirmed in advance using a focus template slide and imager. Generally when an array is spotted between the diamond points on a template, the sleeve will be centered directly above the lens in the slotted stage. The apparatus is not limited to the disclosed preferred embodiments, and other types of devices besides slides or microtiter wells may be utilized for analyte detection with a TOAD™ bioilluminator.

Traditional Translucent Slides

Glass or plastic microscope slides have commonly been used as solid matrix supports for microarray analysis. Probe molecules have been attached to glass or plastic surfaces using cross-linking compounds. (See, e.g., Schena, Microarray Analysis. J. Wiley & Sons, New York, N.Y., 648 pp., 2002.) Probes may be printed as 2D arrays of spots, each of about 100 to 500 μm in diameter. The cross-linking compounds and any coating used to attach probes to the glass surface form a solid matrix, on top of the glass substrate. Many different kinds of cross-linkers are known, depending on the type of reactive moieties (e.g., sulfhydryl, amino, carboxyl, phenyl, hydroxyl, aldehyde, etc.) available on the probe molecules that can be cross-linked to the surface without affecting probe functionality (e.g., target molecule binding).

A problem with previous methods for probe attachment is that the capacity for attachment is limited. As probe size is increased, the number of possible binding sites for prospective target molecules is generally decreased. If the binding sites for the probe are saturated at a level below the threshold for detection, a signal will not be observed even if binding has occurred between probe and target molecule.

Attempts have been made to attach probes to the glass surface using avidin-coated slides and biotin-conjugated probe molecules. Alternatively, silanes, such as aminosilane or 3-glycidoxypropyltrimethoxysilane, have been coated onto the glass surface, with the silane moiety attached to the glass and the reactive moiety cross-linked to probe molecules. Other approaches have utilized slides coated with reactive substrates with functional aldehyde, carboxyl, epoxy, or amine groups that can form a covalent bond with the probe molecules, affixing them permanently to the glass surface.

Although these methods work moderately well for small probe molecules, they tend to work poorly for larger probe molecules (e.g., antibodies) where functionality (binding) may depend on probe orientation, flexibility and degree of cross-linking. Covalent attachment methods also tend to bind very little material to the matrix surface. Consequently, probe concentration is low and signal detection is difficult. Because relatively little probe is available on the surface of the 2D array, such systems show a low signal-to-noise ratio for a positive binding reaction between probe and target.

Protein or peptide target molecules are often detected using antibodies as capture molecules. Two-dimensional arrays used in clinical diagnostics or proteomics frequently utilize antibodies as probes for protein or peptide target molecules. Although antibodies tend to be highly specific for their target antigens, they are not easily attached to glass surfaces with cross-linking agents and standard methods. This is because of the limited amount of material that can be affixed to the matrix with known chemistries, resulting in weak signals generated upon target binding. Another problem is that antibody specific binding cannot be maintained without adequate hydration and support in the matrix. Thus, long term stability of antibody-coupled solid matrix arrays tends to be limited, with inconsistent results obtained depending on the age of the array.

Attempts have been made to solve this problem by creating an environment that stabilizes the protein and preserves its functional probe features. For example, Prolinx Inc. (Bothell, Wash.) has developed a chemical affinity system using standard glass slides with a polymer brush format affixed to their surface. The system relies upon the interaction between two synthetic small molecules that form a stable complex, phenyldiboronic acid (PDBA) and salicylhydroxamic acid (SHA). (E.g., Stolowitz et al., Bioconjugate Chem. 12:229-239, 2001.) PDBA is first conjugated with protein probes. The conjugated probes then link to SHA attached to the polymer brush to form a 3D functional array. This method is limited by the amount of antibody that can be bound to the surface. More importantly, the target antigen must be sufficiently small to diffuse through the brush border in order to react with antibodies affixed to the matrix. Such methods are not suitable for identifying and/or quantifying larger targets, such as whole cells or bacteria.

Opaque Slides

Methods to stabilize and increase the amount of probe attached to matrix arrays are highly desirable. Such methods generally lead to opaque slides, since the matrix materials used to increase probe binding and preserve stability typically involve non-translucent gels, hydro-gels, agars, and other materials coated on the glass surface. Proteins attached to such opaque matrix materials are stabilized by hydrophobic and electrostatic interactions in a three-dimensional array.

Most scanners in current use for genomic and proteomic microarrays read the slides from the same side as the bound probe and target molecules, using opaque matrix arrays. Opaque matrix-coating materials used to produce microarrays include nylon, PVDF (polyvinylidene fluoride) and nitrocellulose. Nitrocellulose, a traditional polymer substrate in use for more than 50 years, is a substrate with very attractive properties for microarray applications. (E.g., Tonkinson and Stillman, Frontiers in Bioscience 7:c1-12, 2002.)

Opaque nitrocellulose has been extensively used to immobilize proteins and nucleic acids for biomolecular analysis. Nitrocellulose immobilizes molecules of interest in near quantitative fashion and allows for short and long term storage. Nitrocellulose also allows for solution phase target species to efficiently bind to immobilized capture molecules. Diagnostic devices using ELISA methods have employed nitrocellulose membranes with a lateral flow process to bind capture reagents to the membrane (Jones, IVD Technology, 5(2):32, 1999).

Traditional opaque membrane materials have a number of attractive features. They are inexpensive to construct, bind more than 100 times the amount of protein that can be bound by linker coated glass slides, and are generally easy to work with. This is particularly true for opaque nitrocellulose membranes, which have a long history of use.

Nitrocellulose is normally produced in a microporous form that may be applied to the surface of glass slides to form an opaque surface. Probes may then be attached to the opaque nitrocellulose membranes in microarrays, using standard nitrocellulose binding methods. Such slides have been used with radioactive, fluorescent and chemiluminescent detection systems (e.g., Brush, The Scientist 14[9]:21, 2000).

Traditional nitrocellulose membranes are also very brittle in the absence of a supporting structure or foundation, leading to frequent cracking or fragmentation. For this reason, opaque nitrocellulose has been used in a microporous form bound to plastic sheets. Such sheets are always opaque, due to the microporous form, and require a supporting structure (e.g. acetate or cellulose) to avoid damage during handling.

Although opaque nitrocellulose membranes exhibit high binding capacity, stability and reasonably low interference with target-capture molecule interactions, they are not suitable for use with optical detection systems designed to work from the opposite side of a matrix array.

Translucent Nitrocellulose Slides

The methods and compositions disclosed herein may be used to produce translucent surface coatings of colloidal nitrocellulose that retain advantageous binding characteristics of opaque nitrocellulose membranes. The interaction between probe and target molecules can be observed directly on a translucent nitrocellulose solid matrix.

In some embodiments of the invention, translucent nitrocellulose matrix arrays may be used in combination with a flow cell, fluidics cube or capillary system (e.g., U.S. patent application Ser. No. 09/974,089). In such embodiments, the translucent nitrocellulose matrix may be attached to one side of a glass or plastic slide. Probes may be attached to the nitrocellulose and the interaction between probe and target molecules observed through the glass with a sensor or camera.

The nitrocellulose material is totally translucent if formed according to the disclosed methods. Light signals may thus be observed without scatter or interference from opaque materials. This allows a greater signal-to-noise ratio and ease of detection of target molecules, compared to opaque microporous nitrocellulose matrix arrays. Such opaque matrix arrays can obscure portions of the light or reaction indicator species (e.g., dye) produced upon binding of target molecules.

Nitrocellulose in the form of a colloid in an amyl acetate solvent has been used by electron microscopists to make castings for specimens. Colloidal nitrocellulose is formed by casting as an ultra-thin film on a water surface. The film may then be picked up on a transmission electron microscopy (TEM) grid and used as a support film for TEM specimens. Because the film must be very clean and uniform, great care is exercised in its production. Colloidal nitrocellulose is readily soluble in amyl acetate. The amyl acetate is water soluble and evaporates evenly to form uniform films. It is supplied as a 1% solution of very pure nitrocellulose.

High purity nitrocellulose in EM grade amyl acetate (Collodion) may be purchased from commercial sources. The amyl acetate is purified by refluxing over calcium oxide to remove all moisture. Soluble and suspended material is removed by slow distillation. The removal of all traces of moisture from the solvent permits the formation of very strong colloidal nitrocellulose films with virtually no holes.

In an exemplary embodiment of the invention, Collodion was obtained in bulk from Ernest P Fullam, Inc. (Latham, N.Y.) and used to manufacture high quality translucent nitrocellulose matrix arrays. An aliquot of 200 μL of 1% Collodion solution was pipetted onto the surface of freshly cleaned standard 25×75 mm glass slides. The Collodion was evenly spread to the edges of the glass slide surface in a dust free area. After drying for 2 hours at room temperature, the slides were heated for an additional hour or more at approximately 60° C. Dried slides were labeled and stored for production of microarrays.

When using a glass array surface, the edges of each slide were sealed with lacquer (e.g. nail polish) or other adhesive to prevent the ultra-thin nitrocellulose substrate from separating from the glass upon exposure to aqueous solutions. When colloidal nitrocellulose is applied to acetate film, nylon slides or other plastic surfaces, it requires no adhesive and binds avidly. Slides may be composed of almost any translucent material as long as the amyl acetate does not react with the surface to discolor it or alter its properties. Certain types of plastics become opaque when exposed to amyl acetate and are not suitable for use with that solvent system. In alternative embodiments of the invention, the colloidal nitrocellulose may be suspended in other volatile organic solvents besides amyl acetate before application to a glass or other translucent slide.

The colloidal nitrocellulose slides may be spotted with probes using any known methods for microarray production. Methods for spotting proteins, peptides, oligonucleotides and nucleic acids onto nitrocellulose surfaces are well known in the art. Antibodies and biotinylated bovine serum albumin were used to determine the colloidal nitrocellulose binding capacity. The estimated protein binding capacity for the initial glass matrix arrays was in the range of 100 to 200 μg/cm².

A CMOS imaging system that detected light emissions through the translucent nitrocellulose matrix arrays was used with a Cy5-streptavidin indicator dye. (See, e.g., U.S. patent application Ser. No. 09/974,089, filed Oct. 1, 2001.) At a loading volume of approximately 5 nanoliters per spot, biotinylated BSA was reproducibly detected at least down to 10 to 20 picograms of protein. Optimal CMOS images were obtained using a protein concentration range of about 20 to 100 μg/mL biotinylated BSA. The CMOS imaging data was confirmed using a 24 hour colloidal gold stain (BioRad, Hercules, Calif.) of the spot arrayed translucent nitrocellulose slides.

The ability of the colloidal nitrocellulose matrix to maintain antibody binding activity was confirmed by spotting a variety of antibodies on the translucent surface. A concentration range of 20 to 200 μg/mL was used. Nonspecific protein binding sites on the nitrocellulose membrane were blocked with 0.1% BSA in buffer solution. The antibodies were then exposed to solutions containing the appropriate target antigen. Binding was detected using biotinylated second antibodies and Cy5-streptavidin indicator.

A 2D array spotted with 5 nanoliters per spot of primary goat anti-mouse antibody solution and was developed using a secondary mouse Listeria monoclonal antibody and a secondary biotinylated goat anti-mouse antibody. In this assay, the primary and secondary goat anti-mouse antibodies bound to different epitopes of the secondary mouse antibody. The two secondary antibodies were thus used to detect primary antibody bound to the array. Using a CMOS imaging system, the primary antibody could be detected down to a level of 100 picograms or less of antibody. The CMOS data was confirmed on the same slides using colloidal gold staining.

An advantage of the translucent nitrocellulose surface is that the progress of the probe binding reaction can be examined by looking through the translucent lower surface of the slide. This allows more effective probe binding to occur. The slide may also be adapted for use with a fluidic cube to mix and deliver samples to the surface. The progress of the probe-target binding reaction may also be monitored in real time through the underside of the slide.

Reconfigurable Microarrays

In certain embodiments of the invention, reconfigurable microarrays may be produced by using small linker molecules, such as aptamers or affibodies, bound to the surface of a solid matrix. Aptamers are oligonucleotides derived by an in vitro evolutionary process called SELEX (e.g., Brody and Gold, Molecular Biotechnology 74:5-13, 2000). Aptamers may be produced by known methods (e.g., U.S. Pat. Nos. 5,270,163; 5,567,588; 5,670,637; 5,696,249; 5,843,653) or obtained from commercial sources (e.g, Somalogic, Boulder, Colo.). Aptamers are relatively small molecules on the order of 7 to 50 kDa. Because they are small, stable and not as easily damaged as proteins, they may be bound in higher numbers to the surface of a solid matrix. This effectively amplifies the number of probe reactive sites on the surface of an array.

Affibody® ligands (U.S. Pat. No. 5,831,012) are highly specific affinity proteins that may be designed and used like aptamers. Affibodies may be produced or purchased from commercial sources (Affibody AB, Bromma, Sweden). Aptamers and affibodies may be used in combination with antibodies to increase the functional avidity of translucent or non-translucent solid matrices for probe molecule binding. Increased binding in turn results in an increased signal strength, greater signal-to-noise ratio, more reproducible target molecule detection and greater sensitivity of detection.

Reconfigurable microarrays may be used in combination with two antibodies and a capture probe. The capture probe may be an affibody, aptamer or any other probe capable of binding one of the antibodies. Both antibodies should selectively bind to a target cell, molecule or antigen.

The effectiveness of binding is increased if the capture probe binds to a portion of an antibody characteristic of the IgG class. Such probes would only require a small part of the antibody structure to be present in order to react and bind to an antibody-target complex. Larger targets, such as microbes or cells are covered with numerous antigens that may form very large complexes with antibodies. However, truncated IgG antibody fragments could interact with such large targets and still bind to an aptamer or affibody probe on the slide surface.

Antibodies are most stable in solution and optimal antigen-target molecule binding occurs with antibodies in solution, not attached to a solid matrix. In preferred embodiments of the invention, the two antibodies may be allowed to bind to the target in solution. Once target-antibody complexes are formed, the complex may be exposed to aptamer or affibody probe molecules on the reconfigurable matrix array. The probes may bind to a first antibody, while the second antibody may be conjugated to a fluorescent tag or other marker. The tagged complex may then be detected on the surface of the matrix array, using optical detection or any other known detection method.

For example, an aptamer may be tailored to specifically bind to the Fc portion of mouse IgG with high affinity. Samples containing target molecules of interest may be allowed to interact in solution with a mouse antibody specific for an antigen of interest. The sample may be mixed with a different biotinylated or otherwise tagged second (non-mouse) antibody that binds to a different epitope on the same antigen. The target antigen bound to the first and second antibodies may be exposed to the aptamer microarray. The anti-mouse aptamer affixes the complex to the solid matrix. After extensive washing to remove unbound tagged antibodies, the complex containing tagged antibody that is attached to the matrix array surface may be detected.

In some embodiments of the invention, multiple analytes may be simultaneously detected on a reconfigurable microarray. Such multiplex assays require that each second antibody specific for a different target antigen be labeled with a distinguishable label. For example, three different second antibodies may be tagged with red, green or blue fluorophores. Using anti-mouse aptamers, mouse antibodies specific for three different targets may be added to a sample and mixed with the target molecules and second antibodies. After exposure to the aptamer array, the presence of each target may be determined by the presence of red, green or blue fluorophores attached to the matrix. The skilled artisan will realize that the invention is not limited to optically distinguishable fluorophore tags and that any known identifiable tag moieties, such as radioactive, fluorescent, luminescent, enzymatic, etc. may be used. The number of target analytes that may be simultaneously detected is limited only by the number of distinguishable tags that may be attached to the second antibody.

The skilled artisan will realize that many variations on this scheme may be used within the scope of the claimed methods. For example, in alternative embodiments of the invention, a first antibody may be used in conjunction with multiple tagged second antibodies, each of which binds to a different epitope of the target molecule. This may occur, for example, where the available second antibodies are polyclonal antibodies. Alternatively, use of more than one second antibody with affinity for the same antigen may improve the sensitivity of detection. In another alternative, one second antibody may bind to a class of targets (for example, all E. coli bacteria) while a second antibody binds to a specific subclass (e.g., E. coli strain O157:H7).

In a non-limiting example, the aptamer detection method may be used to detect microbes in a food sample. An aptamer that exhibits high affinity and specific binding for mouse IgG may be obtained. Such aptamers may be rapidly and readily obtained using SELEX. The anti-mouse IgG aptamer may be attached to a slide using standard methods, preferably with a translucent solid matrix. Non-specific binding sites on the matrix may be blocked and the slide washed before testing.

To detect Listeria monocytogenes, an IgG mouse anti-Listeria m. antibody may be incubated with a food sample of interest at an appropriate concentration (typically 1 to 50 μg/mL). A rabbit (or other non-mouse) biotinylated secondary anti-Listeria m. antibody (1 to 50 μg/mL) and incubated for 5 to 30 minutes. The sample with both antibodies may then be applied to the array containing anti-mouse IgG aptamers. After a short interval (approximately 15 minutes) the array may be washed so that only mouse IgG and rabbit biotinylated antibody complexed with Listeria m. is retained on the array. A solution of Cy5-strepavidin or other indicator applied to the surface may then reveal the presents or absence of an anti-Listeria m. antibody complex affixed to the surface.

A single aptamer with high selectivity for IgG mouse antibodies may be used as a universal extender to capture and detect a variety of microbes. The only requirement would be that the mouse antibody to the microbe or antigen reacts only with the target and the aptamer, while the biotinylated second antibody reacts only with the target and not with the aptamer.

Such an aptamer array is reconfigurable and is dependent only upon the nature of the solutions delivered to its surface. For example, an aptamer array that binds to mouse IgG antibodies may in principal be used to detect any target for which a mouse IgG is available. It is far more cost effective to construct such a reconfigurable array, compared to constructing a series of fixed pattern arrays, each of which may only detect a limited number of targets. Thus, the present invention provides significant cost advantages for use in proteomic and genomic work.

Although the methods disclosed in this section may be used with opaque arrays, they are most effectively used with a translucent matrix where solutions are delivered on one side and examined for reactivity from the opposite side of the array. A fluidic cube, total optical assay device (TOAD™) or the equivalent may be attached to the reactive surface to deliver fluids to various parts of the array, allowing the user to determine which test results will be obtained for a given sample.

Aptamers are more stable than antibodies and large proteins. They are also smaller and bind in higher concentrations to reactive surfaces. Thus, aptamer-based systems extend the avidity of an array for target molecules beyond what could be achieved using larger proteins for array binding. Although the present example is presented with regard to aptamers, the skilled artisan will realize that affibodies or other small ligands could be used in the practice of the claimed methods.

### Total Optical Assay Device (TOAD™)

TOAD™ Bioilluminator

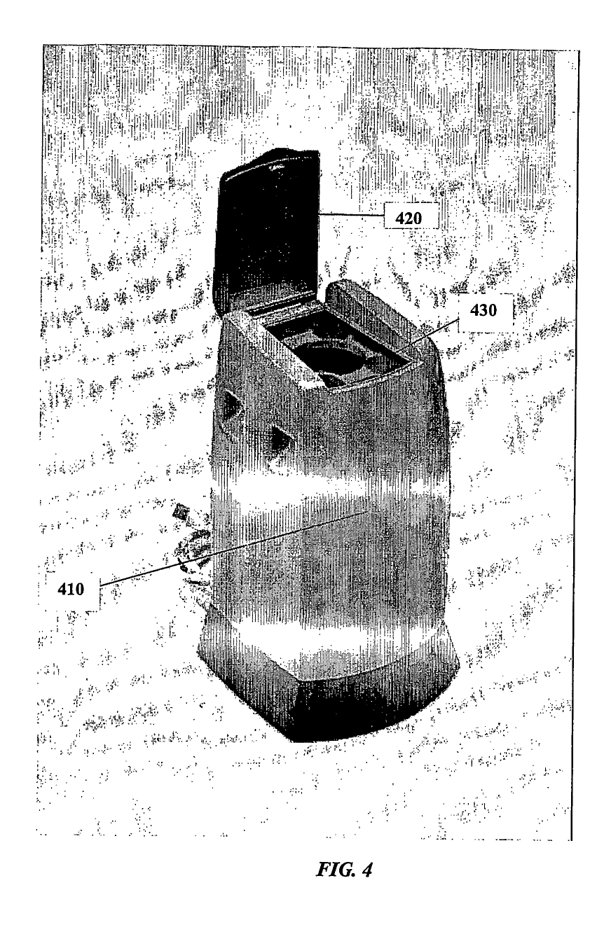

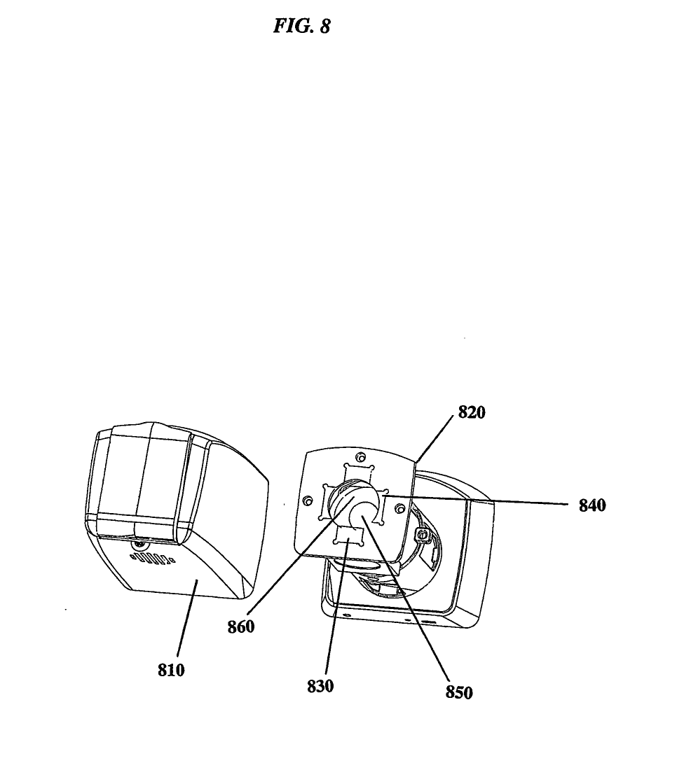

FIG. 4 illustrates a non-limiting exemplary embodiment of a Total Optical Assay Device (TOAD™). In this example, the TOAD™ comprises a light-tight casing 410 with a hinged lid 420. When the lid 420 is closed, the casing 410 blocks external light from the interior of the TOAD™, allowing highly sensitive detection of bioluminescence or other emitted light from samples to be analyzed. The lid 420 is opened to show a stage 430. Devices to be imaged, including but not limited to GRABBER™ slides or FOOTPAD™ microtiter well devices, may be positioned on top of the stage 430. Underneath the stage is a single focusing lens (not shown) that sits on top of a CCD (charge coupled device) chip or other light sensor (not shown). The slide or microtiter wells may be positioned on the stage 430 by aligning them with corresponding shaped depressions (see FIG. 8) on the top of the stage 430. The casing 410 may comprise depressions, handles or other features to facilitate moving the TOAD™.

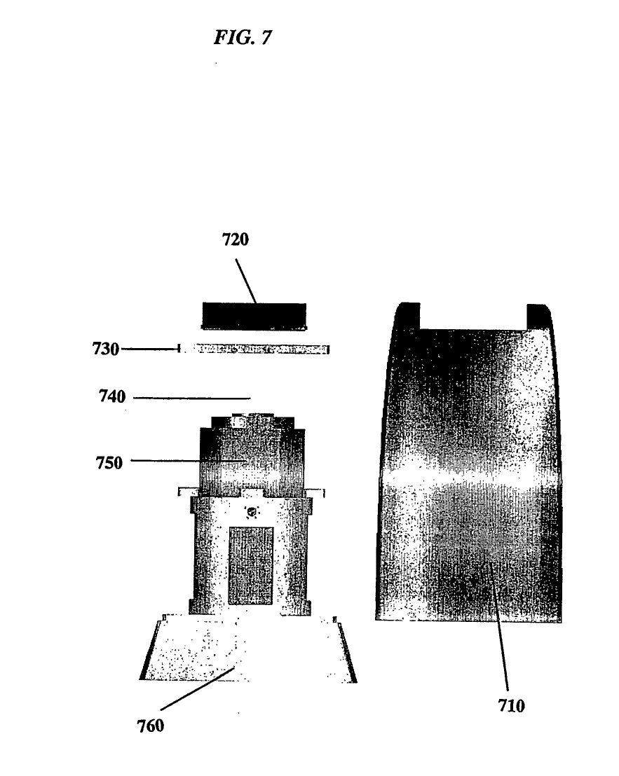

FIG. 7 illustrates a schematic view of an exemplary embodiment of a TOAD™, with the body of the casing 710 displaced to show the internal components. The lid 720 overlies a stage 730, which is used to position slides, microtiter wells or other devices over a CCD chip located at the top of an imaging device 750. In a non-limiting example, the imaging device 750 is a QICAM 10-bit mono cooled CCD camera (Qimaging™, Burnaby, B.C., Canada, catalog #QIC-M-10-C). However, the TOAD™ is not limited to the exemplary embodiment and it is contemplated that other known types of imaging devices 750 may be utilized. A single focusing lens (not shown) is interposed between the CCD chip and the stage 730 and may be attached to the top of the imaging device 750, for example using a C-mount adaptor.

In the exemplary embodiment the imaging device 750 contains, in addition to a CCD chip, a Peltier heat exchange element surrounding the CCD chip. The rest of the imaging device 750 contains electronic components for processing the signals detected by the CCD chip, along with connections for a power supply, software interface and external data port, such as a 6-pin firewire connector. The Peltier heat exchanger removes heat from the CCD camera 750 and releases it inside the casing 710. In order to prevent the TOAD™ from overheating, a separate fan driven cooling device 740 is positioned adjacent to the top of the imaging device 750. The cooling device 740 drives hot air out of the casing through a light-tight vent at the back of the casing 710 (not shown) and circulates cool air through the casing 710 interior. During standard operation, the cooling device 740 operates efficiently enough that the CCD camera 750 may be operated continuously without overheating. Overheating of the CCD camera 750 results in substantial increase in background noise, producing random bursts of apparent light detection throughout the optical field of the CCD chip.

The imaging device 750 rests on top of a base 760 that holds the imaging device 750 in position relative to the stage 730. The bottom of the base 760 is open to allow access to on on-off switch at the bottom of the imaging device 750 and connections to power supplies (e.g. 110 volt AC electrical cord), firewire and any other external connections. A non-limiting list of components that may be incorporated into a TOAD™, including exemplary embodiments and sources of such components, is included in Table 6.

FIG. 8 is a schematic diagram showing the top view of an exemplary embodiment of a TOAD™, with the casing 810 removed to show the upper surface of the stage 820. Indentations 830, 840 are shown in the top of the stage 820, arranged to allow positioning of slides, microtiter wells or other devices over the CCD chip. A hole 860 in the center of the stage 820 allows for light transmission through the slide, microtiter well or other device and detection by the underlying CCD chip 850. It will be apparent to the skilled artisan that the use of transparent or translucent devices, as discussed in more detail below, facilitates the use of a TOAD™ apparatus with an optical detector located below the device to be optically read.

The exemplary embodiment discussed above is designed for optical detection with samples that spontaneously emit light, for example using any known chemiluminescent or bioluminescent detection system, such as the luminol system disclosed below. In this case, no external source of excitatory light is needed to detect bound analytes. The skilled artisan will realize that alternative systems may be utilized, such as fluorescently tagged detection molecules that bind to target analytes. Such systems are well known in the art. The use of a fluorescent detection system would necessitate additional components, such as an excitatory light source (e.g. laser, photodiode array, etc.), cutoff filters to screen the photodetector from excitatory light, and other such known components for fluorescent detection systems. The illustrative embodiment discussed above possesses the advantages of simplicity of construction and use, low cost, and sensitive detection of target analytes with minimal background noise.

Footpads™

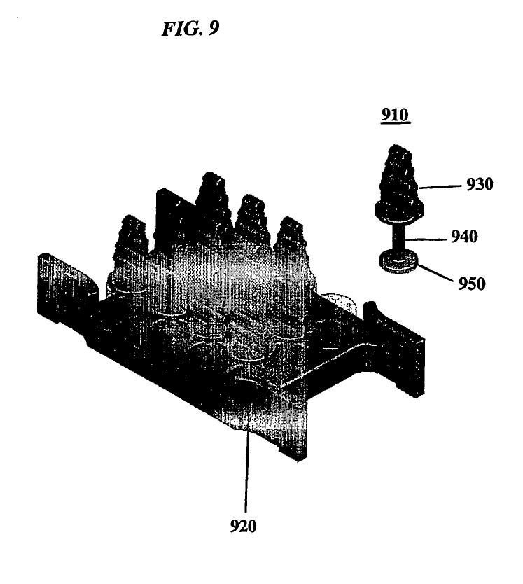

Certain embodiments concern apparatus and methods of use of devices called Footpads™. Footpads™ may come in a variety of designs, an exemplary embodiment of which is shown in FIG. 9. FIG. 9 illustrates an array of 12 Footpads™ 910 contained in a Footpad™ holder 920. A single Footpads™ 910 has been removed from the array to illustrate its structure. The top end 930 of the Footpads™ 910 may be designed for ease of handling, for example with a tapered, ridged shape as shown. A stem 940 may connect the handle 930 to the bottom 950 of the Footpad™ 910. The lower surface of the Footpads™ 910 may be coated with translucent nitrocellulose, rayon, cellophane or other known materials that may bind to probes, analytes or other ligands, as discussed below. The Footpad™ 910 may come prespotted with various probes, calibration spots and other spots, as discussed below. Alternatively the user may load any molecule of interest, such as antibodies or other probes, on the lower surface 950 of the Footpad™ 910.

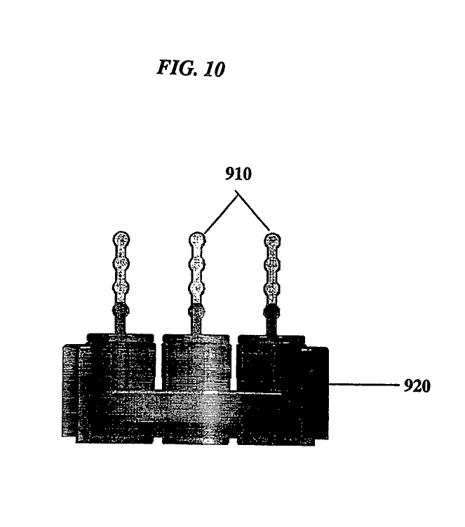

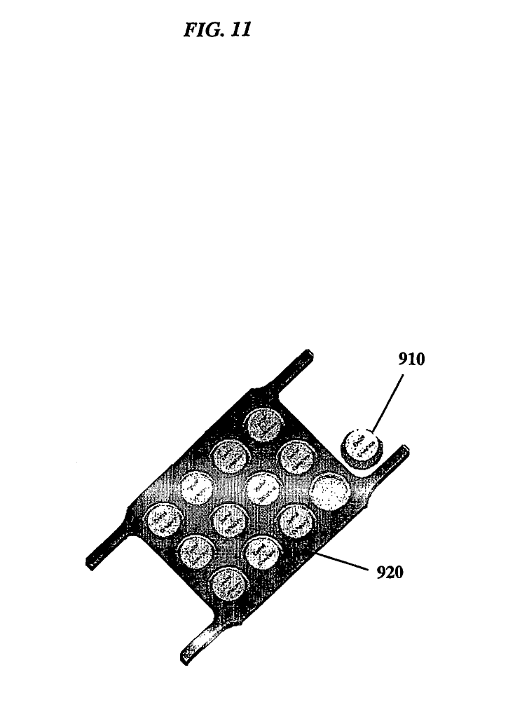

A side view of an array of Footpads™ 910 inserted into a Footpad™ holder 920 is shown in FIG. 10. When inserted fully into the holder 920, the Footpads™ 910 are contained in wells that hold the Footpads™ 910 firmly in place. The spacing of Footpads™ 910 in the holder 920 is designed so that the entire array may be inserted into the wells of a standard 96 well microtiter plate. FIG. 11 shows that the bottom of the Footpad™ holder 920 is open, with space around the rim of the Footpad™ 910 to accomodate the well rims on a microtiter plate. As discussed below, a microtiter plate may be preloaded with reagents and the holder 920 with Footpads™ 910 sequentially immersed in consecutive sets of corresponding microtiter wells. Thus, twelve or more Footpads™ 910 may be exposed to appropriate reagents simultaneously. The holder 920 and Footpads™ 910 may be transferred manually between different sets of wells of a microtiter plate. Alternatively, the holder 920 and/or Footpads™ 910 may be designed to be handled robotically.

Footpad™ Kits

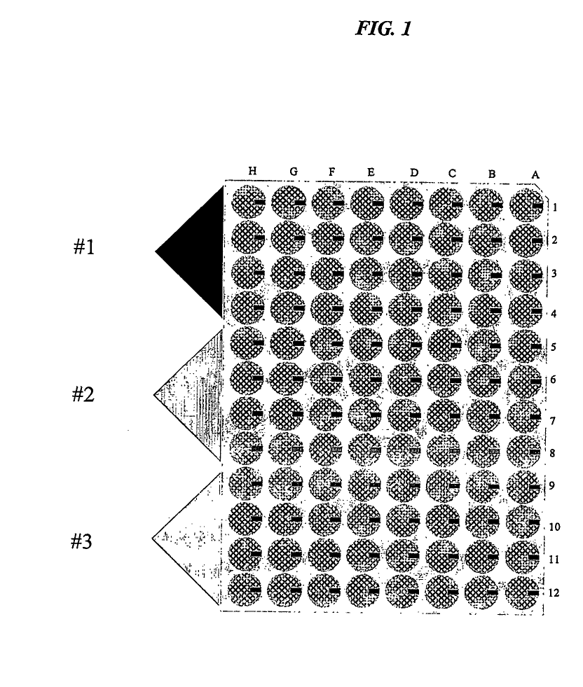

In certain embodiments, a standard 96 well plate with a flat bottom optical surface may be used with pre-loaded reagents and Footpads™ on a bioluminescent TOAD™ detector. A packaged kit may include a labeled, foil sealed 96 well plate with 12 TOAD™ Footpads™ and reagents. Super signal reagents Luminol and peroxide may be mixed immediately before a test and added to the “optical” reservoir H before beginning the test (FIG. 1). The test sample may be placed in the A reservoir (FIG. 1). As indicated in FIG. 1, in certain embodiments, sample is placed in column A wells, blocking solution (100 μL) in column B wells, wash solutions (100 μL) in columns C, E and G wells, antibody cocktail (200 μL) in column D wells, HRP-SA solution (100 μL) in column F wells and luminol solution (100 μL) in column H wells of a 96 well microtiter plate format. The skilled artisan will realize that the disclosed methods and apparatus are not limited to a 96-well format and other formats may be used. Using the 12 Footpad™ holder disclosed above, each column comprising 4 Footpads™ may be sequentially exposed to the solutions in columns A-H of FIG. 1, starting with the first column of 4 Footpads™ immersed in the column A (sample) wells and moving the holder 1 column at a time through the B-H wells. Where solutions are present in less than all 8 columns of the microtiter plate, as discussed below, the Footpad™ holder may be manipulated accordingly.

Luminol-peroxide reagent is a solution containing luminol and peroxide that is prepared and used on the same day tests are initiated. Luminol in the presence of HRP (horse radish peroxidase) and peroxide forms an unstable intermediate that produces light (see below). In the process it is consumed. Because it is in abundant excess relative to the amount of HRP trapped in a microspot on the surface of a slide or in a microplate well, light emission may be sustained for several hours. The Luminol and peroxide may be dispensed and mixed in equal volumes. The solution is then diluted using dilution buffer equivalent to the total volume in the first mixture (50% dilution). For example, if 1 ml of Luminol is combined with 1 ml of peroxide, then 2 ml of dilution buffer may be used to dilute the reagent to produce 4 mL of solution for testing.

A stage may be fitted to the TOAD™ detector to accommodate the 96 well plate (see FIG. 8). The stage should securely fix the microplate so that 4 of the “H” reservoirs (FIG. 1), which span 25 mm, fit the optical window of the instrument. In a 96 well microplate format, the first 4 “optical” H1, 2, 3, and 4 reservoirs (FIG. 1) are positioned over the target area. The well plates are then shifted in increments of 4 “optical windows” so that alignment and imaging can accommodate all 12 of the H reservoirs in 3 steps. The three positions of interest are shown and labeled in the diagram as position #1, #2, or #3 (FIG. 1).

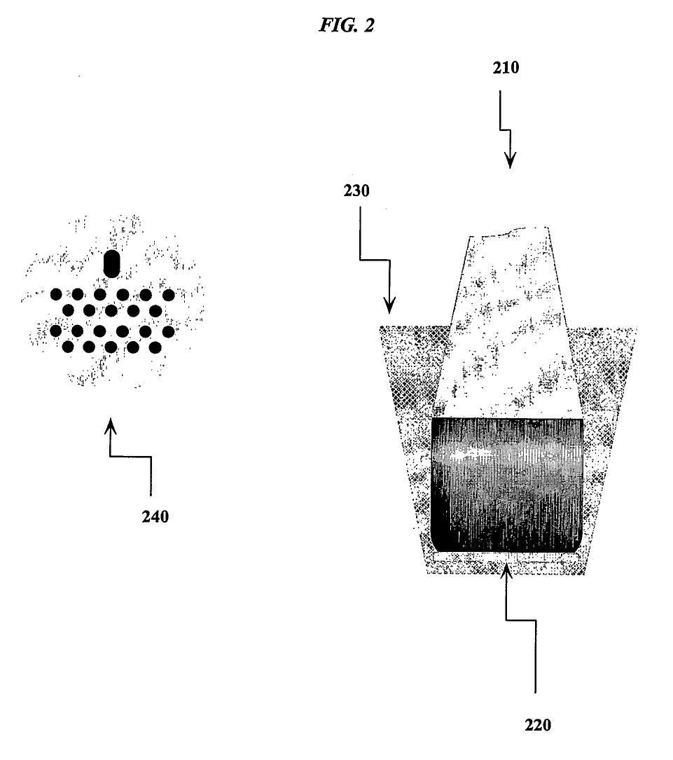

Embodiments involving plates pre-loaded with reagents and Footpads™ sealed with foil allow the operator to conveniently run from 1 to 12 test samples by loading only luminol-peroxide and sample into the microplate. Each row 1 through 12 represents a single sample process. With a breakaway row and seal the packaged kit can be used for incremental tests until all 12 rows are consumed, providing the stage adjusts to lock the “optical” well H on the target imager. Each Footpad™ in an arrayed format accommodates up to 24 spotted probes (FIG. 2) and allows for the possibility of multiplexing a single sample. FIG. 2 illustrates an exemplary embodiment, with an Eppendorf pipet tip 210 affixed to the top of a Footpad™ with a probe binding surface on the bottom 220, the Eppendorf tip surrounded by a reservoir with fluid 230. With a 96-well format, the Footpad™ surface 220 is about 7 mm in diameter. An exemplary pattern of capture antibodies (200 μM spots) and index spot is also shown 240.

Using a “blank” non-arrayed Footpad™ in a 96 well microplate with an empty “antibody cocktail” reservoir allows the user to test for any targets of interest. This is accomplished by either manually spotting primary antibodies onto a pre-treated Footpad™, or affixing a robotically spotted cellophane, translucent nitrocellulose, rayon or other array of spots to the Footpad™. The operator then fills the empty reservoir with the biotinylated secondary antibodies, the H reservoir with luminol-peroxide, and the A reservoir with sample for testing (FIG. 1).

Since 96 well plates are easily obtained, inexpensive, and optically suitable for a bioluminescent assay, and because preloading the plate is straight forward, one format for the bioluminescent TOAD™ may include a stage to accommodate the 96 well plate in the manner indicated.

A variety of samples may be analyzed using the methods and apparatus disclosed herein, including virtually any type of environmental, clinical, veterinary, pathologic or medical sample. In different embodiments, samples may be analyzed after collection without any sample treatment whatsoever. In other embodiments, samples may be processed after collection and before analysis. Samples may be processed in various ways, including without limitation cooling, freezing, heating, homogenization, organic phase extraction, detergent extraction, enzymatic digestion, centrifugation, filtration, ultracentrifugation, ultrafiltration, lyophilization or various well known chromatographic procedures. In certain embodiments, it is anticipated that solid samples may be treated by homogenization and, if necessary, crude filtration, such as filtering the sample through a nylon or other filter with a pore size of approximately 100 μm or less.

Exemplary Footpad™ and Spotting Pattern

In certain embodiments, TOAD™ Footpad™ are cylinders measuring 7 mm in height and 7 mm in diameter, fitting with the lower flat coated surface resting on the flat bottom of the well reservoir (FIG. 2). The exposed upper surface may be used to affix an inverted eppendorf pipet tip, handle or appendage so that the pad may be moved easily from one reservoir to the next. The lower surface of the Footpad™ is coated with an appropriate binding medium and may be either blank or pre-arrayed (spotted) with capture antibodies (probes). Each pad may be marked with an index calibration spot (FIG. 2) serving two purposes.

The index when placed in luminol-peroxide provides a reference for luminosity based on a standard spot. It also serves to identify any other spots that react in a test since the position relative to the index is always known. It does not matter if the pad rotates in the reservoir, since capture probe spots are identified in spatial relationship to the index calibration spot. All positions are correctly readable as long as the pad is placed face (treated surface) down in a reservoir.

A total of 24 spots may be affixed to a single Footpad™ without exceeding a density of 100 spots per square cm. A binary ID code may be affixed to a Footpad™, for exampole using the first two rows of spots, and used to provide a tracking lot ID number.

Translucent Nitrocellulose Binding Surface Affixed to the Footpad™

The lower surface of the Footpad™ may be used to accept and firmly affix probes. The composition of the surface may be important for optimized probe binding and analyte detection and quantification. Depending upon how the array or spotting is done, the composition may vary. In preferred embodiments, the lower surface of the Footpad™ is coated with a thin layer of translucent nitrocellulose.

Colloidal 1% nitrocellulose firmly affixes to nylon, and to a lesser extent to other plastics and glass. As shown herein, the translucent nitrocellulose surface formed after drying on these surfaces avidly binds proteins and other substrates, while preserving their functionality (e.g., enzyme activity, antibody-antigen binding, etc.) A Footpad™ of nylon to which translucent nitrocellulose has been applied may be used. This format works well for manual spotting on the pad but may be less useful for robotic arrayers, which are mostly designed to print on ordinary slides measuring 25×75×1 mm.

In some embodiments, a sheet of nylon coated with nitrocellulose may be used to print the array. The array may then be punched out and affixed to the lower surface of the Footpad™ with an adhesive. In alternative embodiments, a device may be constructed to hold coated Footpads™ firmly in position for robotic array work. Additional details of embodiments using translucent nitrocellulose layers for probe binding are disclosed below.

Rayon and Cellophane Substrates

We have shown that 100% rayon thread, when immersed in a phosphate buffer with biotinylated BSA, affixes the B-BSA and produces a strong signal easily seen in the HRP-SA Luminol-peroxide format on the bioluminescent detector. The signal is intense and specific for biotin affixed to the thread, as can be demonstrated by first blocking the thread with 0.1% BSA. No signal is evident if the thread is blocked first before adding HRP-SA and Luminol-peroxide. It appears that rayon, a naturally occurring cellulose polymer, behaves in a manner similar to nitrocellulose. Untreated “raw” cellophane is identical to 100% rayon and can be obtained in translucent thin sheets. It is abundant and inexpensive.

In some embodiments, functional Footpads™ to bind probes may be manufactured out of rayon. Alternatively, cellophane printed discs may be affixed to the Footpad™ with an appropriate adhesive. The array may be printed on a thin sheet of cellophane before affixing it to glass, plastic, or any other solid support.

The Footpad™ may be of any color and does not have to be translucent. A rayon or cellophane surface requires no solvent or drying step, making the manufacturing process less complicated than the colloidal nitrocellulose process for coating either slides or Footpads™.

96 Well Microplate Procedure

In the following discussion, HRP-SA reagent refers to horse radish peroxidase conjugated with streptavidin in a solution formulated to enhance antigen detection. The solution is normally stable for at least 2 years from the time of formulation. Its activity can be verified in a test by placing one drop into 100 μL of luminol-peroxide solution. In a totally dark room, the faint blue light emitted shows that the light-producing reaction has begun.

The Footpads™ are either precoated and spotted with probes or precoated and blank (no spots on the pad), placed in the empty reservoir A column (FIG. 1). The operator using a blank (non-pre-arrayed) package will conduct assays with probes and biotinylated antibodies that are provided by the user. Because the Footpad™ comes already prepared for manual spotting, the operator needs only to remove the Footpad™, apply spots to its lower surface, fill the antibody D reservoir (FIG. 1) with biotinylated antibodies, fill the luminol-peroxide H reservoir (FIG. 1), block the Footpad™, and begin the test.

Material List Using Pre-Coated Blank Footpad™ Test

The following materials may be used for a pre-coated blank Footpad™ test.

Pre-coated blank Footpad™ 96 well kit

Spotting buffer

Primary antibody for target of interest

Secondary biotinylated antibody to form the sandwich immunocomplex

Antibody dilution buffer with stabilizer

Index marking solution

Luminol-peroxide reagent

Sample for testing

TOAD™ bioilluminator

Desktop computer and software

Material List Pre-Arrayed Blank Footpad™ Test

For pre-arrayed Footpad™ tests, the following materials may be used.

Pre-arrayed Footpad™ 96 well kit

Luminol-peroxide reagent

Sample for testing

TOAD™ bioilluminator

Desktop computer and software

Pre-Coated Blank Footpad™ Procedure

All reagents and materials may be assembled and identified before initiating a test. In this embodiment, the user has elected to use antibodies for probes and secondary biotinylated antibodies to capture a target when using a blank Footpad™. The procedure is illustrated in Table 1.

Pre-Arrayed Footpad™ Procedure

All reagents and materials may be assembled and identified before initiating a test. In this embodiment, the user has elected to use a pre-arrayed Footpad™ configuration, making the entire process considerably simpler and faster using spotted Footpads™. Only two solutions are loaded into well reservoirs. The luminol-peroxide reagent is mixed and added to reservoir H (FIG. 1). The sample is placed in reservoir A (FIG. 1). Processing begins after the Footpad™ has been adequately blocked, incubating 5 minutes in reservoir B. The procedure is illustrated in Table 2.

Component Configuration

Exemplary Kit Components of Potential Use with a Footpad™ Format are Indicated in Table 3.

Binary ID Code

A binary code of spots may be used as an index to track production batches of pre-arrayed Footpads™, GRABBER™ slides or other items. An exemplary method of coding is disclosed herein. A binary code system to identify a production particular lot may be used in the first two rows of the Footpad™ (FIG. 2). There are 3 reference spots in the first calibrator position. The reference spots are used to determine if the other spots in the adjacent rows and column are binary 1 or binary 0. A 4^{th} spot located at the coordinates for row 2 column 2 is a quality assurance check sum on the intended code for the lot number assigned. The other spots in the first two rows are either empty or filled with the biotin marker at the same concentration and volume as is used for calibration spots and are determined to be either 0 or 1 by comparison to the 3 binary reference marks.

Binary 0 Reference Spot

The binary 0 reference spot contains 1/10^{th} of the concentration of the biotin marker used in a standard calibration spot. If any of the spots used to set the binary code are measured with a luminosity of lower intensity than the binary 0 reference, they are assigned a value of 0. A spot measured with a greater luminosity than both the binary 0 reference and the binary 1 reference is assigned the value of 1. A spot with a measured luminosity in between the binary 0 and binary 1 reference spots registers as a defective Footpad™ and is reported as an error to the operator.

Binary 1 Reference Spot

The binary 1 reference spot contains 3/10^{th} of the concentration of the biotin marker used in a standard calibration spot. If any of the spots used to set the binary code are measured with a luminosity of greater intensity than the binary 1 reference, they are assigned a value of 1. A spot of lower luminosity than both the binary 1 reference and the binary 0 reference is assigned the value of 0. A spot measuring luminosity between the reference spots registers as a defective Footpad™ and is reported as an error to the operator.

Binary Code and Lot Number Assignments

For each production of a Footpad™ there is a lot production date. The date of production may serve as the Lot number assignment for that production. The date may be printed in a binary code format. The unique pattern for a particular production date may be determined when a request for a lot production run is made.

A proprietary software program coding and assigning filled and empty spots may be used so that a user by entering the date for the production run will see the spots that are to be filled and those to be left empty. The program used for the pattern of filled spots in the ID code also generates the assignment for the check-sum validation reference.

Check Sum Validation Spot

A reference spot at row 2 column 2 may be either completely filled or left empty as a validation of the binary code on the Footpad™. If the sum of the value 1's in the binary code assigned for a particular date is an even number, then the value 0 (empty spot) may be assigned for the check sum validation spot. If the sum of the value 1's in the binary code assigned for a particular date is an odd number, then the value 1 (filled spot) may be assigned for the check sum validation spot. The check sum validation when read against the binary code reference spots will be either 0 or 1. If the value read fails to match the assigned value for a particular production date, the Footpad™ will be assumed to have been printed incorrectly and discarded.

GRABBER™ Slides

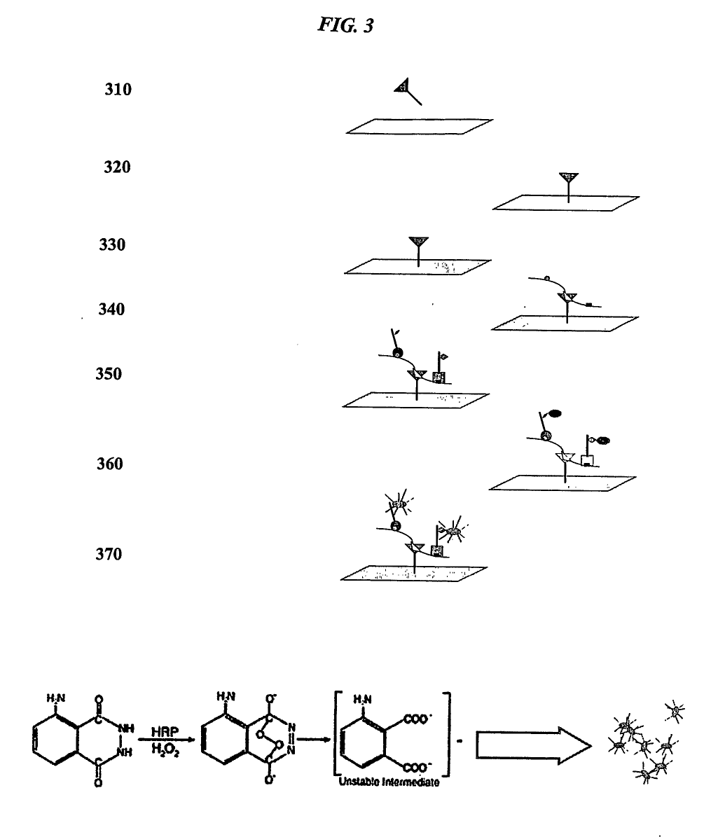

Embodiments of the invention concerning a microtiter well format are discussed above (Footpad™). Alternative embodiments concern devices and methods of use of translucent coated slides (GRABBER™) (FIG. 3). FIG. 3 illustrates an exemplary protocol for use with GRABBER™ slides, comprising affixing a primary antibodies to the slide 310, drying the 2D array 320, blocking the slide 330 (10 min), capturing antigen with the primary antibodies 340 (10 min), forming an immunocomplex with secondary biotinylated antibodies 350 (10 min), forming HRP-SA complexes 360 (10 min), adding luminol reagent 370 and capturing an image (10 min). The luminescent reaction catalyzed by HRP is as indicated in the bottom of FIG. 3.

GRABBER™ slides provide for an easy to use method for detecting and identifying one or more targets of interest in a solution assayed on the surface of a slide. Slides may be provided pre-spotted with capture probes (e.g., antibodies) affixed ready for immediate testing. Alternatively, users who want to prepare their own 2D-spotted array may in 1 hour prepare a manual array for testing.

In preferred embodiments, the method of detection utilizes a primary antibody capturing an antigen target to form an immunocomplex with a second antibody that is biotinylated or otherwise labeled. The first antibody probe is affixed to a translucent nitrocellulose slide (GRABBER™) and during the entire procedure remains affixed to the surface of the slide.

The immunocomplex, if formed, may be detected using a well-known enzyme activated bioluminescent process. Horseradish peroxidase (HRP) catalyzes the breakdown of peroxide and produces an intense light if luminol is added to the solution during the breakdown process. Luminol forms an unstable free radical intermediate that results in the release of photons at 430 nm (FIG. 3). By conjugating HRP to streptavidin (SA), the immunocomplex may be detected with an appropriate and sensitive photon detector. Biotin and streptavidin rapidly combine to form this light producing complex.

Typical protein (e.g., antibody) probes may be spotted with 1 to 5 nL per spot at a concentration of 0.05 to 0.2 μG/mL. Where primary antibodies are attached to a slide, matrix array or other surface, secondary tagged antibody, such as biotinylated antibody, may be provided in a dilution buffer premixed at an optimized concentration for target detection with pre-spotted slides. The optimized concentration may be determined empirically by the user. Secondary antibodies may be diluted in dilution buffer to a concentration typically in the range of 5 to 30 μG/mL.

A TOAD™ (total optical assay device) may be provided with proprietary software to allow the user the means to read, interpret and record signals at the surface of a slide. An image file and report may be saved or printed for subsequent analysis and comparisons.

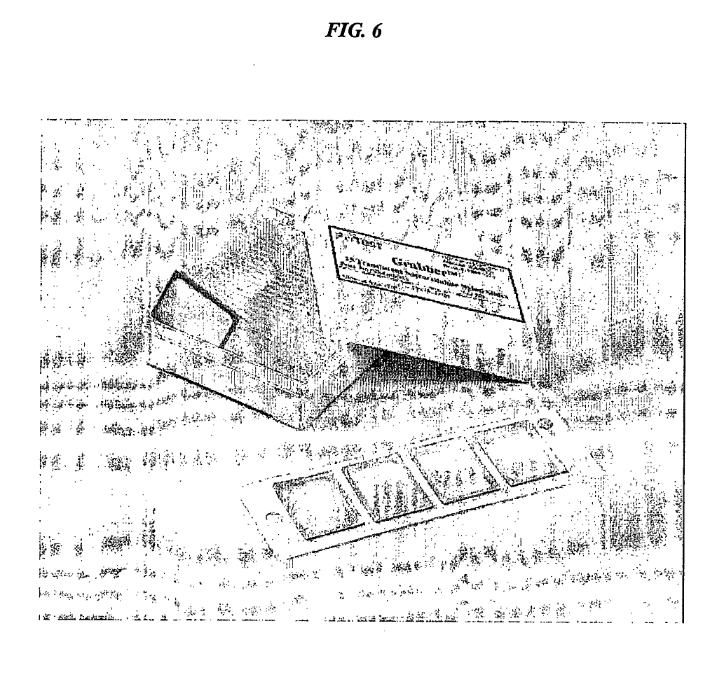

The user may provide a sample that is placed in an incubation sleeve (FIG. 6) affixed to the slide and allowed to react for 10 minutes before it is discarded (see below).

GRABBER™ slides may be coated with a translucent nitrocellulose substrate (see U.S. patent application Ser. No. 10/373,546, filed Feb. 24, 2003) that avidly and immediately binds proteins, carbohydrates and/or oligonucleotides, strongly affixing them to the coated surface while preserving the functionality and 3D structure of the bound molecule. No other chemical process is required to affix the probes to the slide surface, making the 2D array procedure simple and fast. The hydrophobic surface with a superior contact angle is well suited for compact robotically printed arrays.

The bioluminescent reaction is long lasting and the signal may be acquired over prolonged periods, allowing for extraordinary sensitivity in detection. Many targets may be simultaneously identified in a single sample. The proper primary probe antibody and biotinylated secondary antibody should strongly interact with the target for adequate detection. Preprinted slides and reagents provided are optimized to produce high quality detection.

Materials List

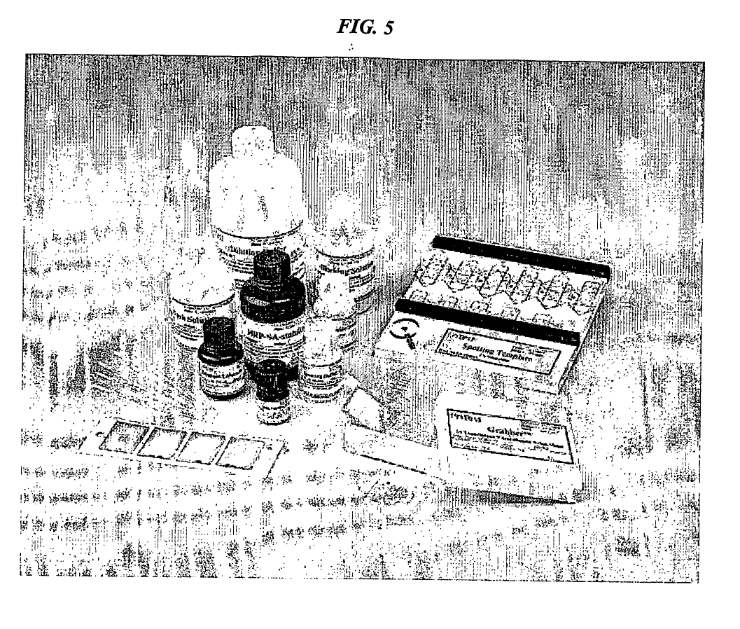

All of the disposable slides, reagents and other materials needed to assay for analytes using GRABBER™ slides with a TOAD™ bioilluminator may be provided in kit form, as illustrated in FIG. 5. FIG. 6 shows an exemplary set of GRABBER™ slides, along with an incubation sleeve for performing the binding, washing and other procedures on the slide. The exemplary incubation sleeve shown in FIG. 6 contains four separate reaction chambers for use with GRABBER™ slides.

The following exemplary materials may be used to perform a pre-coated nitrocellulose slide assay. Non-limiting exemplary kit materials for use with GRABBER™ slides are also listed in Table 7, along with exemplary sources for the materials. A kit may be provided with all the materials needed to complete an assay. The user may assemble gloves, forceps, a timer, waste container for spent material, and eppendorf tips for spotting an array. A sample for testing should be acquired before beginning the assay. If the array is pre-spotted, testing may begin with the blocking as shown in FIG. 3.
GRABBER™ pre-coated slide
Spotting buffer
Spotting template
Focus Template slide
Primary antibody for target of interest
Secondary biotinylated antibody to form the sandwich immunocomplex
Antibody dilution buffer with stabilizer
Index marking solution
HRP-SA reagent
Luminol-peroxide reagent
Sample for testing
Incubation sleeve
TOAD™ bioilluminator
Desktop computer and software
Disposable dispensers
Timer
Dust free latex gloves
Forceps
Pre-Spotted Array Protocol

All reagents and materials may be assembled and identified before initiating an assay. In certain embodiments, pre-spotted arrays ready for testing are obtained. Slides have incubation sleeves (FIG. 6) affixed to the nitrocellulose surface and secondary biotinylated antibodies are provided ready for use. An exemplary protocol for use with pre-spotted arrays is disclosed in Table 4.

Protocol for User Prepared 2D Array

In an alternative embodiment, the user may elect to provide primary antibodies for probes and secondary biotinylated antibodies to detect a target when using a pre-coated non-arrayed slide. An exemplary protocol is shown in Table 5.

Detection Unit

The discussion above illustrates the use of a TOAD™ containing an exemplary CCD based optical detection system. The apparatus and methods are not limited to the disclosed preferred embodiments. In certain embodiments, a TOAD™ may comprise one or more alternative detection units. Optical signals may be produced when analytes bind to probes and HRP-conjugated secondary antibodies in the presence of luminol, as disclosed above. The signals may be detected by the detection unit. The detection unit may comprise one or more detectors, such as a spectrometer, monochromator, CCD device, CCD camera, photomultiplier tube, photodiode, avalanche photodiode or any other device known in the art that can detect an optical signal. An optical signal may comprise any form of electromagnetic radiation, emission, or absorption, although in preferred embodiments the optical signal comprises visible light.

In preferred embodiments, the presence of analyte attached to one or more probes is indicated by a bioluminescent spot on the surface of the slide, 96-well microplate or other surface. In various embodiments, the device may comprise a data analysis and storage unit, such as a computer or microprocessor. Suitable devices are well known in the art and the present invention is not limiting as to the type of data analysis and storage unit used. The data analysis and storage unit may be operably coupled to the detection unit, so that optical signals are collected, processed and stored. In alternative embodiments, the data analysis and storage unit may store information on each sample collected, including the sample source, geographical location and any other data collected on the sample. The unit may further identify each analyte detected in the sample and store that data as well. In certain embodiments, the device may comprise an interface for downloading data to a remote location, either directly (for example by an incorporated radio transmitter or modem) or indirectly (for example, by downloading to diskette or other storage device or by printing a hard copy).

In certain embodiments, one or more optical components may be interposed between the spot containing surface and detector, such as a lens or lens array to focus optical signals from each spot. A non-limiting example of a detector may comprises a CMOS camera sensor or equivalent unit, such as a PixeLink model AL633 CMOS imager (Vitana Corp., Ottawa, Canada).

In an exemplary embodiment, a nylon slide or other matrix array may be secured on a stage. A CMOS imager may be used to capture the light signals. The CMOS chip may be located beneath the slide and aligned so that spots on the slide are directly above the imager and are sharply focused on the imager surface with optical lenses and apertures. Additional details of a CMOS imaging system and data analysis method of use are disclosed in U.S. patent application Ser. No. 10/373,408, by Lawrence R. Green, entitled “Image Sensor Optimization,” filed on Feb. 24, 2003, incorporated herein by reference.

Probes

Various embodiments concern the use of probes for the detection of analytes. Although in preferred embodiments the probes are antibodies, it is contemplated that virtually any molecule or aggregate that can bind to a target analyte with sufficient affinity and specificity to allow its detection may be used. Standard procedures for the production of monoclonal or polyclonal antibodies are known (see, e.g., Harlow and Lane, Antibodies: A Laboratory Manual, Cold Spring Harbor Laboratory, Cold Spring Harbor, N.Y., 1988; incorporated herein by reference).

Polyclonal antibodies are prepared by immunizing an animal with an immunogen and collecting antisera from the immunized animal. A wide range of animal species can be used for the production of antisera. Typical animals used for production of anti-antisera include, for example, rabbits, mice, rats, hamsters, pigs or horses. Because of the relatively large blood volume of rabbits, a rabbit is a preferred choice for production of polyclonal antibodies.

Antibodies, both polyclonal and monoclonal, may be prepared using conventional immunization techniques, as will be generally known to those of skill in the art. A composition containing antigenic epitopes of an analyte can be used to immunize one or more experimental animals, such as a rabbit or mouse, which will then proceed to produce specific antibodies against the analyte. Polyclonal antisera may be obtained, after allowing time for antibody generation, simply by bleeding the animal and preparing serum samples from the whole blood.

A given composition may vary in its immunogenicity. It is often necessary, therefore, to boost the host immune system, as may be achieved by coupling an immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating an analyte to a carrier protein are well known in the art and include glutaraldehyde, m-maleimidobenzoyl-N-hydroxysuccinimide ester, carbodiimide and bis-biazotized benzidine.

The immunogenicity of a particular immunogen composition can be enhanced by the use of non-specific stimulators of the immune response, known as adjuvants. Exemplary and preferred adjuvants include complete. Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies, depending upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified composition comprising an analyte. The immunizing composition is administered in a manner effective to stimulate antibody producing cells. Cells from rodents such as mice and rats are preferred, however, the use of rabbit, sheep or frog cells is also possible.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus (Kohler and Milstein, Nature, 256:495-497, 1975; Eur. J. Immunol., 6: 511-519, 1976), and those using polyethylene glycol (PEG), such as 37% (v/v) PEG, have been disclosed (Gefter et al., Somatic Cell Genet., 3: 231-236, 1977). The use of electrically induced fusion methods is also appropriate (Goding, In: Monoclonal Antibodies: Principles and Practice, 2d ed., Academic Press, Orlando, Fla., pp. 60-61, and 71-74, 1986).

Fusion procedures usually produce viable hybrids at low frequencies, around 1×10⁻⁶ to 1×10⁻⁸. However, the viable, fused hybrids may be differentiated from the parental, unfused cells by culturing in a selective medium that contains an agent that inhibits cell growth for unfused cells. Exemplary agents are aminopterin, methotrexate, and azaserine. Where aminopterin or methotrexate are used, the medium is supplemented with hypoxanthine and thymidine as a source of nucleotides (HAT medium). Where azaserine is used, the medium is supplemented with hypoxanthine.

A preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three wk) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines also could be cultured in vitro, where the mAbs are naturally secreted into the culture medium from which they can be readily obtained in high concentrations. Monoclonal antibodies produced by either means may be further purified, if desired, using filtration, centrifugation, and various chromatographic methods such as HPLC or affinity chromatography.

Although the methods and compositions disclosed herein allow for the production of novel antibodies against analytes, it is contemplated that previously characterized antibodies or commercially available antibodies may be used to bind to and detect any analyte of interest.

Non-antibody probes that may be used include, for example, aptamers (e.g., U.S. Pat. Nos. 5,270,163; 5,567,588; 5,670,637; 5,696,249; and 5,843,653, incorporated herein by reference), peptide libraries (e.g., U.S. Pat. Nos. 5,565,332, 5,596,079, 6,031,071 and 6,068,829, incorporated herein by reference), and various receptor proteins, binding proteins, cell surface proteins, and other non-antibody peptides or proteins known in the art.

Probe Labels

In various embodiments of the invention, labeled probes may be prepared by any methods known in the art. In certain embodiments, a label moiety may be incorporated into a probe (e.g., peptide, protein, oligonucleotide) during synthesis. In other embodiments of the invention, labels may be attached by covalent, noncovalent, ionic, van der Waals, hydrogen bonding or other forces following probe synthesis. Methods for attaching fluorescent or other labels to probe molecules are known in the art and any such known method may be used to make labeled probes within the scope of the present invention. In particular embodiments of the invention, a probe molecule may be biotinylated and may bind to an avidin or streptavidin-conjugated fluorophore. Fluorophores and conjugated fluorophores may be obtained from commercial sources, such as Molecular Probes, Inc. (Eugene, Oreg.).

Labels of use may comprise any composition detectable by electrical, optical, spectrophotometric, photochemical, biochemical, immunochemical, or chemical techniques. Labels may include, but are not limited to, conducting, luminescent, fluorescent, chemiluminescent, bioluminescent and phosphorescent labels, chromogens, enzymes or substrates. Fluorescent molecules suitable for use as labels include, but are not limited to, dansyl chloride, rhodamineisothiocyanate, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red. A variety of other known fluorescent or luminescent labels may be-utilized. (See, e.g., U.S. Pat. No. 5,800,992; U.S. Pat. No. 6,319,668.)

Methods of Optical Signal Analysis

According to an embodiment of the invention, images such as images for bioanalyte detection may be captured by an imager. The signal data may be corrected for sensitivity variation in the imager. The corrected data may then subjected to a leveling process.

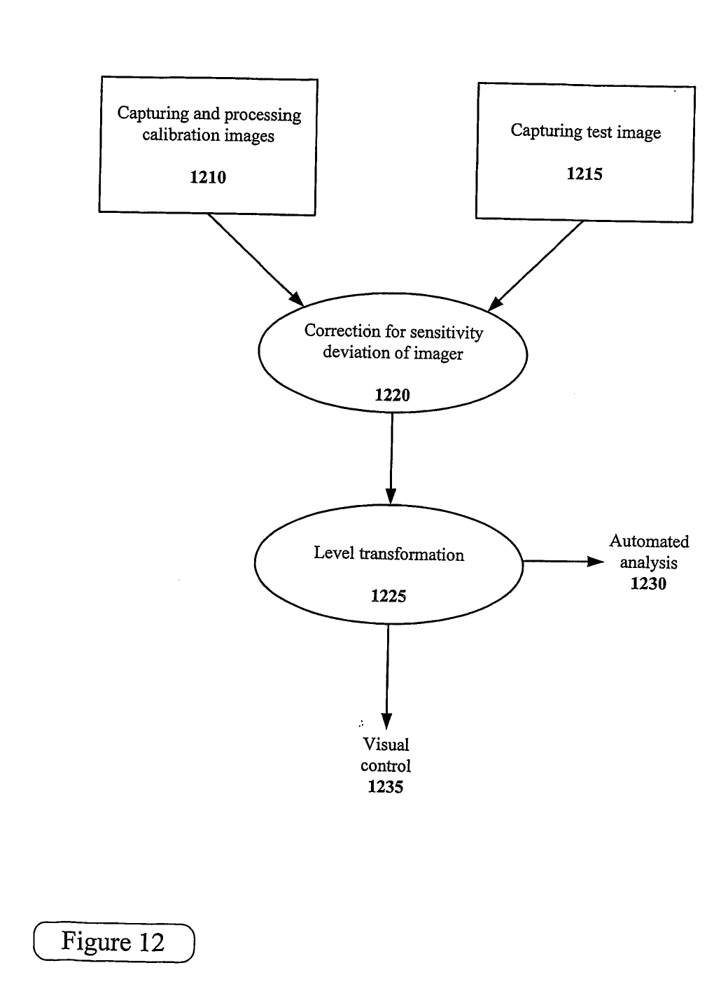

FIG. 12 illustrates an embodiment of an imaging operation 1205. Under a particular embodiment of the invention, the operation provides for imaging bioanalyte detection. In this illustration, the process begins with capturing and processing calibration images 1210. The process of capturing and processing calibration images provides for a calibration of the imaging system to adjust for differences in sensitivities for pixels or regions of the imager. In calibration, a number of images, such as images 1 through k, are captured. Each of the k images has a known exposure level for calibration. In one embodiment, the k images have varying exposure levels that represent an even distribution over a particular range of exposure levels. The calibration process is performed at least once for an imager, but may be performed as often as needed.

Using the data obtained from the k images, an interpolation function is created between exposure levels and average pixel signal values. The function may be expressed as F_Average(exposure). The interpolation function provides an average signal value expected for any exposure level in the range, based on interpolation between the average results of the calibration images. For example, if an exposure level x produces an average interpolated pixel signal level of y, this may be expressed as:
F_Average(x)=y [1]

An interpolation function may also be created between exposure levels and the resulting signals produced by each pixel of the imager. The function for the nth pixel of the image may be expressed as Func[n](exposure). The interpolation function provides a signal value expected for a pixel for any exposure level in the range, based on interpolation between the results of the calibration images for each pixel. For example, if an exposure level x produces a pixel signal level z for the nth pixel of the image, this may be expressed as:
Func[n](x)=z [2]

The results produced by the average interpolation function F_Average (exposure) and the interpolation functions for each pixel Func[it] (exposure) may be based on linear interpolation. However, the optical signal analysis is not limited to any particular method of interpolation. A different method of interpolation may be implemented and may provide more precise results in certain circumstances.

A test image may be captured 115. The image may be captured using a particular test exposure level (TestExposure). The capture of the test image results in the production of a pixel signal value for each pixel in the imager. The pixel signal values provide data regarding the radiation reaching the imager. However, the sensitivity of the pixels may vary. Under an embodiment of the invention, the process provides for correction of sensitivity deviation of imager areas 120. For each pixel of the imager, a correction coefficient may be calculated. The coefficient for a pixel is equal to the average interpolation function shown in equation [1] divided by the interpolation function for the pixel as shown in equation [2]. For pixel n and exposure level TestExposure:
Coefficient[n]=F_Average(TestExposure)/Func[n](TestExposure) [3]

While this embodiment provides for a coefficient for each pixel, other embodiments may provide for correction coefficients that are based on different regions or groups of pixels.

A corrected test signal for the nth pixel may then be obtained by multiplying the received test signal (TestSignal[n]) by the coefficient for the pixel obtained from equation [3]:
TestSignalCorrected[n]=Coefficient[n]×TestSignal[n] [4]

The corrected data may then be provided for automated analysis 130. The corrected data may also be subjected to level transformation to improve the visual control of the test results 125. Under an embodiment of the invention, level transformation for a test image may include creation of a histogram of the image that reflects the number of pixels that provide each corrected pixel signal value. An end-in-search contrast stretching method may then be applied to the histogram. In an embodiment of the invention, this method includes applying a constant percentage value P to saturate the image to full white and black. The method then provides for parsing through the histogram to find the high and low thresholds for the image. A particular signal depth value (such as 255, reflecting an eight-bit value) may be utilized. The leveled signal (LeveledTestSignalCorrected) for each pixel may be produced by comparing the corrected test signal with the High and Low thresholds. If the corrected value is less than the low threshold, the leveled signal is set to zero. If the corrected value is greater than the high threshold, the leveled signal is set to the depth value, such as, for example, 255. If the corrected value is between the high and low thresholds (or equal to the high or low threshold), the corrected value is set to the depth times the difference between the corrected signal value and the low threshold divided by the difference between the high and low thresholds.

Under an embodiment of the invention, assuming that Low is the Low threshold and High is the High threshold obtained from a histogram, the production of the leveled signal for the nth pixel may be expressed in the following pseudocode:
IF TestSignalCorrected[n]<Low THEN LeveledTestSignalCorrected[n]=0
ELSE IF Low←TestSignalCorrected[n]←High THEN LeveledTestSignalCorrected[n]=0
ELSE LeveledTestSignalCorrected[n]=Depth

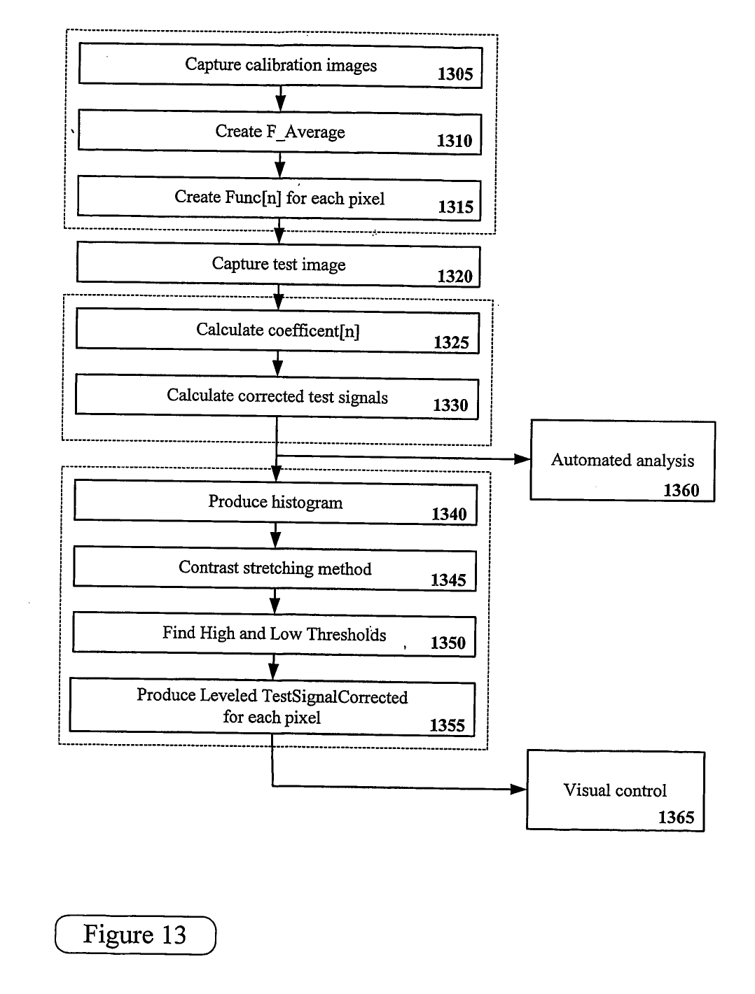

FIG. 13 is an exemplary flowchart illustrating an embodiment of an imaging process. The embodiment shown in FIG. 13 assumes that a calibration is made of an imager followed by the capture of a test image. In this embodiment, calibration (1305-1315) includes capturing a certain number of calibration images 1305 using an imager. The calibration images are of varying exposure levels, with pixel values being captured for each such calibration image. An interpolation function (F_Average) is created 1310 to produce an average interpolated signal value for any exposure level. An interpolation function is created 1315 for each pixel (or region) of the imager to produce an interpolated signal value for any exposure level.

Upon calibrating the imager, a test image may be captured 1320. The capture of the test image produces signal values for each pixel of imager, but such values are based upon the varying sensitivity of each pixel. Under an embodiment of the invention, the captured signal values are corrected for sensitivity (1325-1330). A coefficient is calculated for each pixel 1325, with the coefficient being the average interpolation function value for the test exposure level divided by the pixel interpolation function for the test exposure level, such as shown in equation [3]. Corrected signals are calculated for each pixel 1330, with the corrected signal equaling the test signal produced by the pixel times the coefficient for the pixel, such as shown in equation [4]. The corrected signals then may provided to any automated analysis 1360.

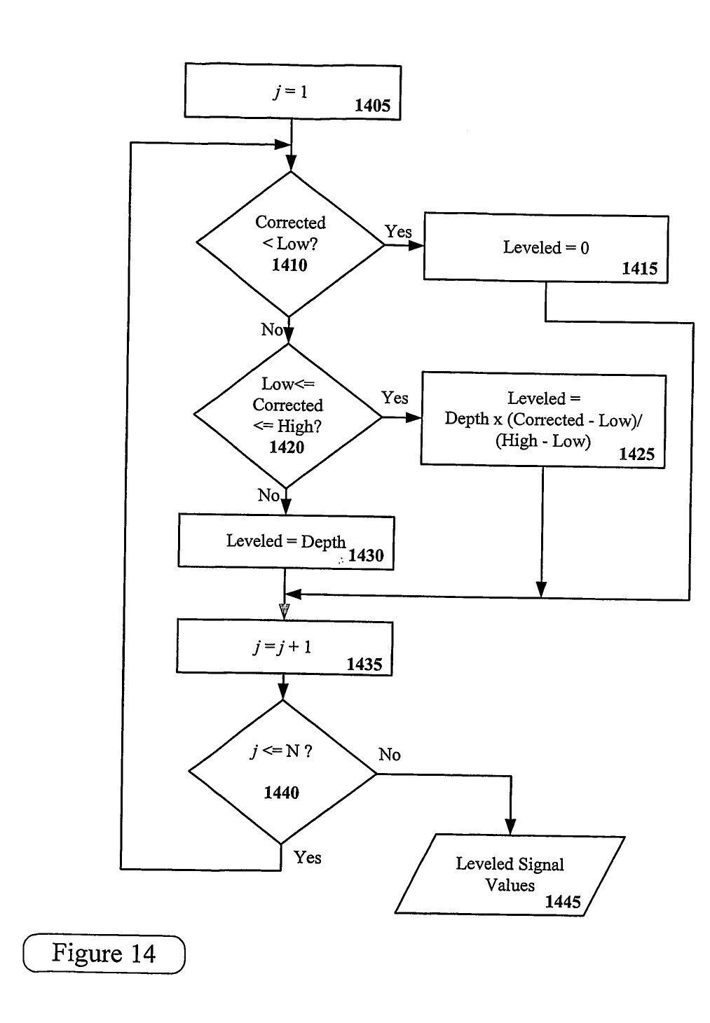

Under an embodiment of the invention, the corrected signal values may be subjected to a level transformation (1340-1355). A histogram is produced 1340 based upon the relation between each corrected pixel signal value and the number of pixels producing such corrected pixel signal value. A contrast stretching method is applied 1345, which may include a constant percentage being saturated to full white and black. The histogram is then searched to determine the High threshold and Low threshold for the histogram 1350. Using such data, a leveled corrected test signal is produced for each signal 1355, which is shown in more detail in FIG. 14. Upon producing the leveled data, the data may be provided to visual control 1365.

FIG. 14 is an exemplary flowchart illustrating an embodiment of producing leveled corrected test signals for an imager. In this illustration, it is assumed that there are N pixels, which, for simplicity of explanation, are numbered 1 through N. For illustration, j is a counter variable used in the process. The counter j is initially set to 1 (1405). For the jth pixel, there is a determination whether the corrected signal value is less than the Low threshold 1410. If so, the leveled signal value is set to zero 1415. If not, there is a determination whether the corrected signal is between (or equal to either of) the Low threshold and the High threshold. If so, the leveled value is set to the depth value for the imager times the difference between the corrected signal value and the Low threshold, divided by the difference between the High threshold and the Low threshold 1425. If not, the corrected signal value is greater than the High threshold and the leveled value is set to the depth value for the imager 1430.

The counter j is then incremented 1435 and there is a determination whether the counter value is less or equal to than the number of pixels N 1440. If so, the process continues for the next pixel 1440. If not, then all pixels have been processed and the leveled signal values have been produced 1445.

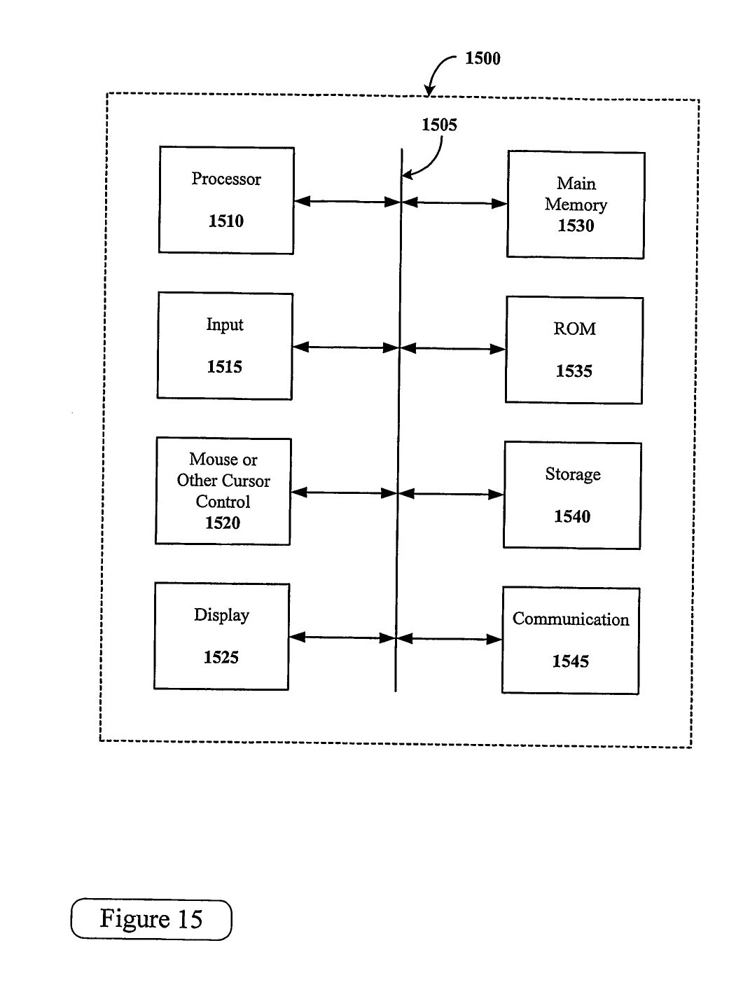

FIG. 15 is block diagram of an exemplary computer 1500 that may be utilized in connection with an embodiment of the invention. FIG. 15 contains a possible embodiment of a computer, but embodiments of the invention may be implemented using many different general and special purpose computers.

Under an embodiment of the invention, the computer 1500 includes a bus 1505 or other for transferring data and communicating. The bus may include multiple buses utilized for different purposes. The computer 1500 includes a processor 1510 to process information. The processor 1510 may include one or more individual processors or coprocessors.

The computer 1500 may include an input 1515 coupled with the bus 1505. The input is used for communicating information and providing commands. The input 1515 may include a keyboard, a keypad, a touch-screen, a voice-activated process, or other input devices. The computer may include a mouse or other cursor control 1520 to control cursor placement and to provide commands. The computer 1500 may include a display 1525, such a CRT (cathode ray tube) or LCD (liquid crystal display) screen to display information. The display 1525 may include a device to produce sound, such as a speaker.

The computer 1500 includes a main memory 1530, such as random access memory (RAM), to store data. The computer 1500 may include read only memory (ROM) 1535 or other similar storage for storing static data. The computer 1500 may include data storage 1540, such as a magnetic disk (hard disk or floppy disk), an optical disk (CD-ROM or DVD), flash memory, or other memory systems.

The computer 1545 may include communication 1545. The communication 1545 may include a wired or cable interface (including but not limited to Firewire, USB, parallel, and serial cables), a wireless interface, a network interface card, a radio transceiver, or other interface. The computer 1500 may be connected to a network or to other computers, devices, or instruments using the communication 1545.

Analysis using Image Sensors

The data analysis scheme disclosed above is a non-limiting example of methods of data analysis of use for analyte detection with the disclosed apparatus. In alternative embodiments, different methods of data analysis may be utilized, either alone or in conjunction with the above-disclosed method. The data analysis methods disclosed below are of particular use where detected light signals are not monochromatic.

When used for analytic purposes, the accuracy of an image sensor in detecting and recording the photon flux striking the image sensor is important. The use of an image sensor to produce visually pleasing results is not important for analysis, but is a design factor of modern image sensors that may be used for analytic purpose. Under an embodiment of the invention, signals generated by an image sensor as a result of light striking the image sensor are optimized to produce data that more closely represents the actual photon flux. The outputs of pixels of the image sensor are normalized to correlate with photon flux.

Image Sensor Operation

Investigators using a photodiode detector may incorrectly assume that the digital data acquired from the detector correlates with the photon flux striking the detector because increases in the intensity of the signal out will generally directly correlate with increases in signal input in a particular wavelength or band-width. Because photodiodes are very linear in output, increases in the photon flux at different wavelengths over the photodiode surface will, over the photodiode's dynamic range, produce a linear output signal. However, the output data will not correlate with the photon flux if the QE is variable over the range of wavelengths that are striking the photodiode. For a particular number of photons striking a photodiode over a time period, a larger current will be produced by the photodiode at a first wavelength than a second wavelength if the QE for the photodiode is higher for the first wavelength than the second wavelength.

Restricting a light source for analysis to a narrow band by filtering or by using a laser light source generally will not resolve accuracy issues. Emission spectra that are evaluated using image sensors may be very broad even if the excitation source has a narrow wavelength range. For this reason, the shape of the QE curve for a photodiode should be carefully considered in evaluating output data from an image sensor.

With regard to the choice of image sensors, CMOS imager sensors are fundamentally different from charge-coupled devices and are increasingly used in microscopy and diagnostic instruments because they are cheaper to build and require considerably less power to operate. CCD cameras are no longer clearly superior in low intensity light situations, which had been true in the past.

CMOS images now rival traditional color imaging methods on film and are easily manipulated. The images may be transferred from one processor to another as a digital file in a variety of formats preserving the arrayed data pixel address. Manufacturers have devoted considerable energy reproducing “life-like” color image sensors using various color filters and interpolation methods to enhance a digital image rendering colors very close to the human eye experience. However, the pleasing “life-like” color pictures obtained with a color CMOS image sensor are not as useful for analytic procedures. Similar issues exist with monochromatic images unless the image is produced by light at single wavelength, which is rarely true.

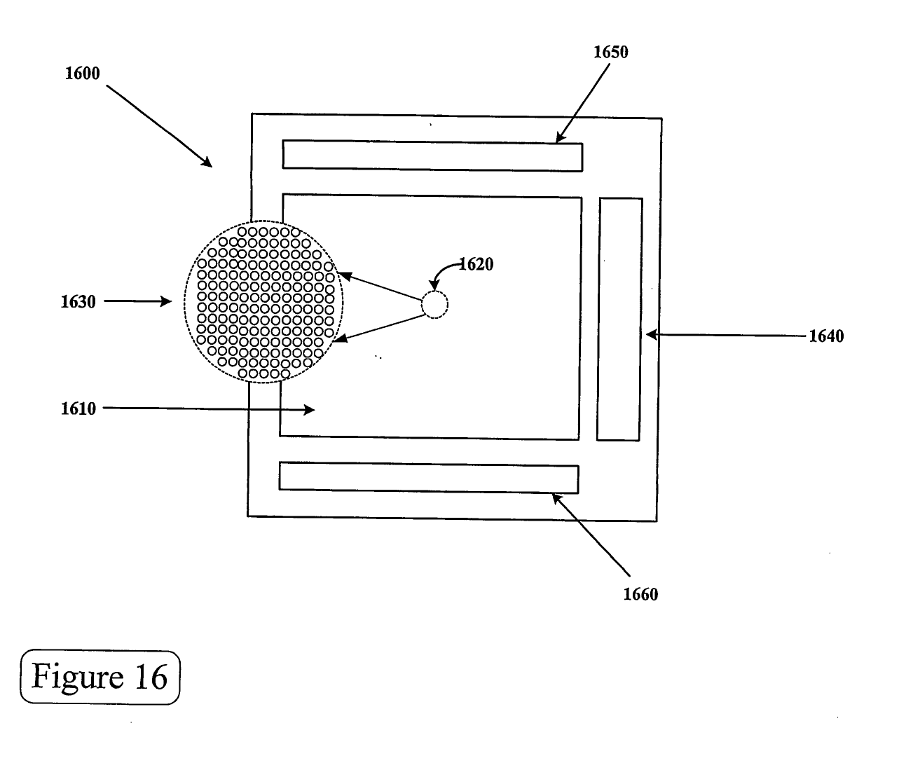

FIG. 16 is a simplified illustration of an example of a color CMOS image sensor that may be utilized in connection with an embodiment of the invention. Not all components and features of CMOS imagers are illustrated. FIG. 16 and the remaining figures below are for illustration and are not necessarily drawn to scale. In FIG. 16, an image sensor 1600 includes an imaging array 1610. The imaging array is comprised of a large number of pixels arranged in a two-dimensional array. As shown in the magnified pixel array 1620 of a particular area of the array 1620, there is a filter associated with each of the pixels in the imaging array 1610. The image sensor 1600 will also generally contain electronics relating to the processing and transmission of signals generated by the imaging array 1610, including analog signal processing 1640, analog to digital conversion 1650, and digital logic 1660.

The photodiode array in a CMOS color image sensor is blanketed by an ordered thin layer of polymeric filters, such as in a conventional Bayer RGB (red-green-blue) two-dimensional array. Each filter is sized to fit over an individual photodiode in a sequential (Bayer) pattern to capture color information from a broad bandwidth of incident illumination. In an RGB array, a heavy emphasis is placed on the green filters to address the human visual maximal response at 550 nm. There are 2 green filters for every red and for every blue filter. However, even though the human eye is more attuned to the green 550 nm region, yellow is generally a better choice with regards to QE factor.

CMOS image sensors and the integrated circuits that define the active pixel array are inherently monochromatic (black and white) devices that respond only to the total number of electrons striking the photodiodes, not to the color of light. Color is detected either by passing the light through a sequential series of filters (such as red, green, and blue filters), or with miniature transparent polymeric thin-film filters that are deposited over the pixel array.

Active pixel sensor (APS) technology is the most popular design for CMOS image detectors. In addition to a photodiode, each pixel (or imaging element) includes a triad of transistors on its surface that convert accumulated electron charge to a measurable voltage, reset the photodiode, and transfer voltage to a vertical column bus. The photodiode thus occupies only a fraction of the pixel area. The photodiode area encompasses an area equal to 30 to 80 percent of the total pixel area for most CMOS sensors. This area occupied by the photodiode is the area that absorbs photons, while the other parts of the pixel are relatively opaque, blocking, reflecting, or absorbing light. The photodiode area or window is referred to as the “aperture” or “fill factor” of the pixel or image sensor. A small aperture or fill factor results in a significant loss of sensitivity and a corresponding reduction in the signal to noise ratio and leads to a reduction in the dynamic range of the sensor.

CMOS image sensors can be utilized to produce pictures based upon the signals produced when photons strike the photodiode surface associated with each pixel in an active pixel sensor array. The pixel signals are processed to form the total picture either in monochrome (black and white) or color.

Monochrome CMOS imager sensors do not have color filters over the photodiode portion of the pixel. However, color CMOS imagers, even with standard Bayer pattern filters, generally are more sensitive than monochromatic CMOS imagers. While it may appear that inherently monochromatic CMOS photodiode without filters would be more sensitive because some light passing through a filter is absorbed and never reaches the photodiode in a color filtered photodiode, this is not generally true. This assumption does not fully take into account the effect a filter has on the QE for a photodiode, which might enhance certain signals, and ignores the advantages provided by microlenses in color photodiode architecture, which are described below. Monochromatic CMOS image sensors do not have a color filter and they do not normally have a microlenses over each pixel. These are important factors that make monochromatic imagers less attractive than color CMOS image sensors with regard to imager sensitivity.

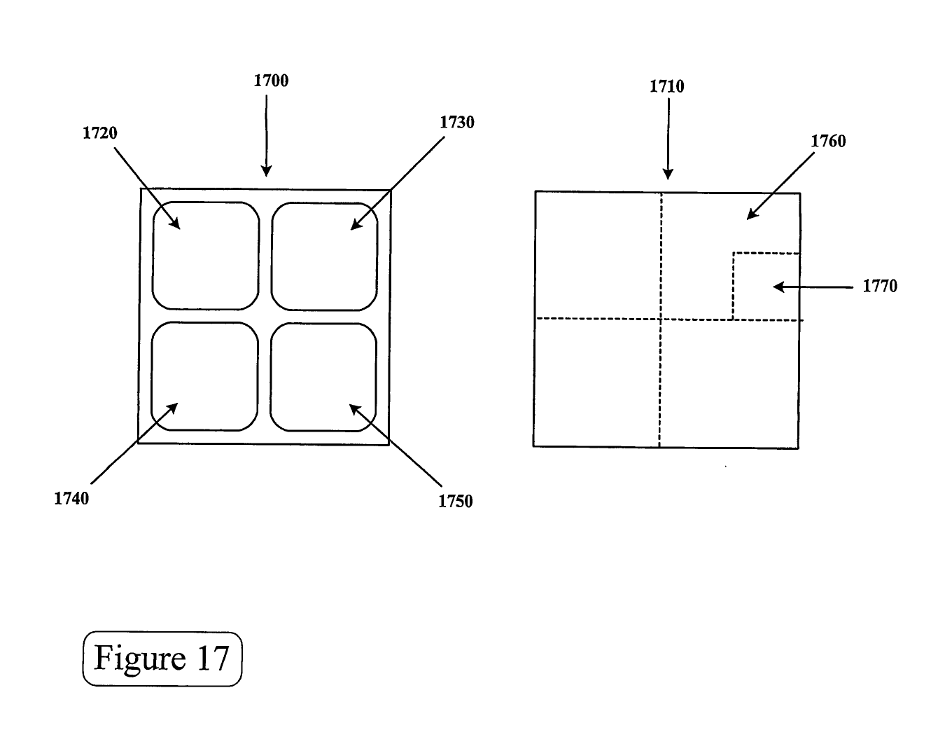

FIG. 17 illustrates a small section of an imaging array of a color image sensor that may be utilized in connection with an embodiment of the invention. The illustrated section repeats throughout the imaging array. The section 1700 is comprised of four pixels, each having a filter. The filters 1720-1750 have colors based on the choices made for the array. Below the filters 1710 illustrates the structure of the individual pixels. For each pixel, such as pixel 1760, there is a portion that comprises the photodiode 1770, the area of the photodiode being only a fraction of the total area. The image sensor will detect only the portion of the light falling on the photodiode area.

In color imagers used in analysis, one possible approach would be to construct imagers by carefully selecting filters and photodiodes to produce QE factors for a given bandwidth that is approximately constant. By combining an appropriate number of photodiodes in an array with chosen filters, the measurement of light energy would be more accurate. The filters chosen could assist in leveling and improving upon the QE factors. However, in practice the filters and photodiodes are chosen for other purposes, with a goal of producing the most visually pleasing images. In order to improve upon the quantum efficiency and spectral response, several CMOS manufacturers use color filter arrays based on the primary subtractive colors, cyan, magenta, and yellow (CMY), instead of the standard additive primaries red, green, and blue (RGB). CMOS image manufacturers generally use either Bayer RGB or Bayer CMY patterns that have been selected for photographic imaging.

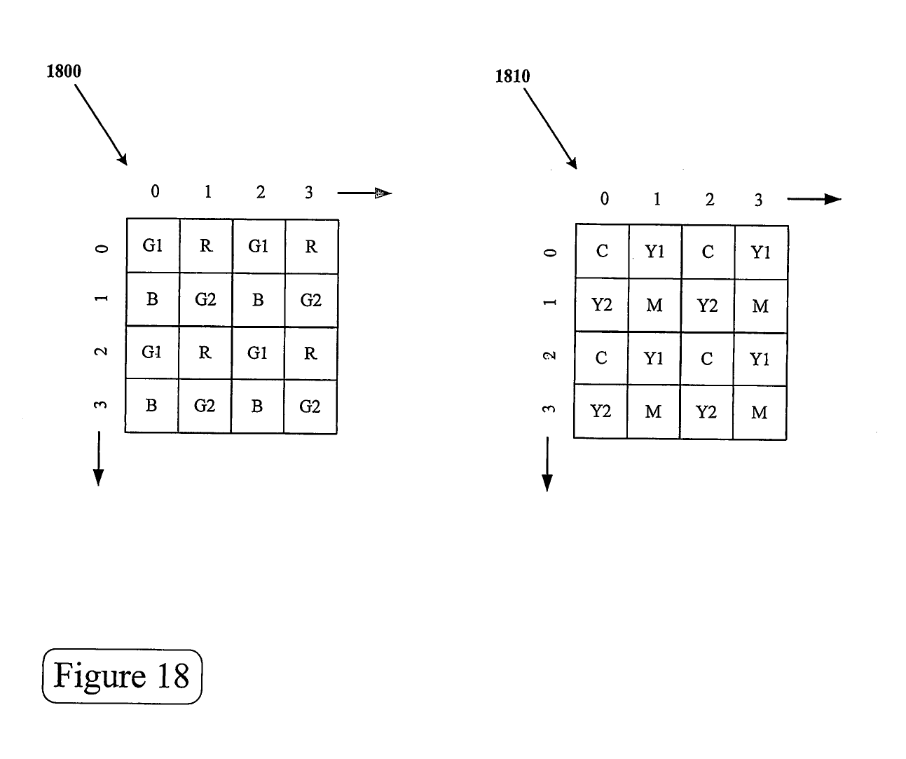

FIG. 18 is an illustration of RGB and CMY filters. For these filters, an imaging array is divided into small arrays of filters, with each such array of filters having the same filter pattern. An RBG filter array 1800 contains two-by-two arrays of filters, with each array containing a red filter and a blue filter for two diagonal pixels and two green filters for the remaining two diagonal pixels. A CMY filter array 1800 also contains two-by-two arrays of filters, with each array containing a cyan filter and a magenta filter for two diagonal pixels and two yellow filters for the remaining two diagonal pixels. While the illustrated filters are the most common filter arrangements, many other filter colors and patterns are possible, and any filter pattern may be used in conjunction with an embodiment of the invention. Certain other alternative filter patterns that provide benefits in certain wavelength ranges are shown in Table 8.

TABLE 8
Alterative Color Filters for a CMOS Color Imager

| Wavelength Range (nm) | Filter Type | Modified Bayer Pattern |
|---|---|---|
| 510-810 | Yellow | Y, Y, Y, Y |
| 610-810 | Magenta | M, M, M, M |
| 490-550 | Cyan | C, C, C, C |
| 490-810 | Yellow, Cyan | Y, Y, Y, C |
| 350-550 | None | Monochrome pattern |

In contrast to monochrome image sensors, color CMOS image sensors also contain microlenses that effectively direct photons to the photodiode aperture. The bubble lens, generally including an anti-reflective coating, can effectively increase the surface area of a photodiode by a significant amount, approximately 60 percent in certain applications. The microlenses substantially increase the effective fill factor and may more than compensate for filters that cut down on the total light that can reach the photodiode.

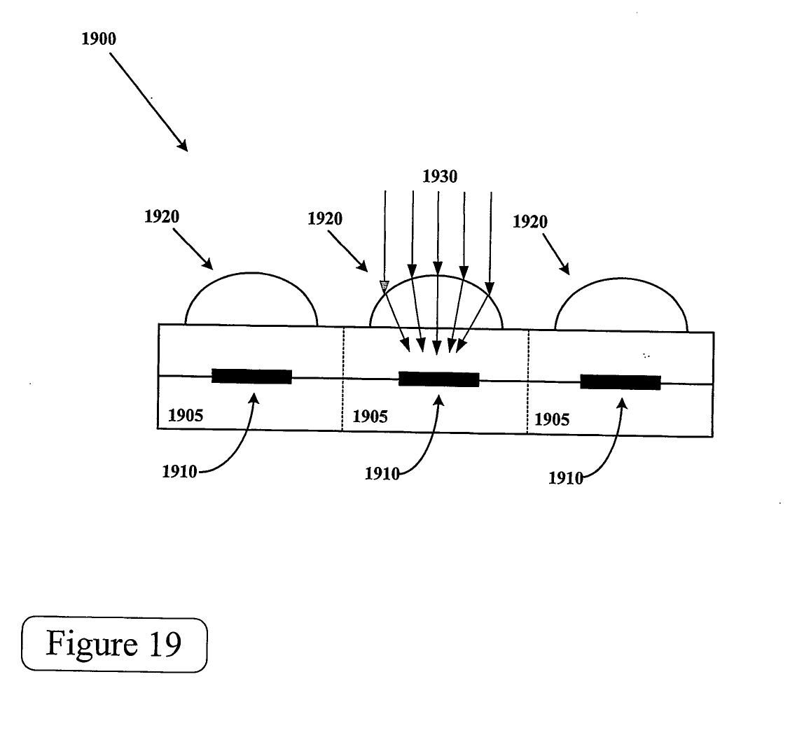

FIG. 19 is a simplified illustration of microlenses in an image sensor. Within the image sensor, there are multiple pixels 1905. Each pixel contains an active portion 1910, with the active portion including only a portion of the pixel area. In order to compensate in part for the light energy that would not normally strike any of the active portions 1910, each of the pixels 1905 has an associated microlens 1920. The function of each microlens 1920 is to focus more light energy on the active portion 1910 and thus to allow measurement of a larger percentage of the incident photon flux. For example, light 1930 strikes a microlens 1920 and is focused on the active portion 1905 of the pixel 1910.

Image Sensor Optimization

Three primary mechanisms that reduce or hamper photon collection by the photosensitive area of an image sensor are absorption, reflection, and transmission. These factors are wavelength-dependent in nature, and define in part the quantum efficiency (QE) of the image sensor. For example, reflection and transmission of incident light occurs as a function of wavelength, with a high percentage of shorter wavelengths below 400 nm being reflected. Shorter wavelengths are absorbed in the first few microns of the photosensitive region but the longest wavelengths exceeding 650 nm often pass through the photosensitive region.

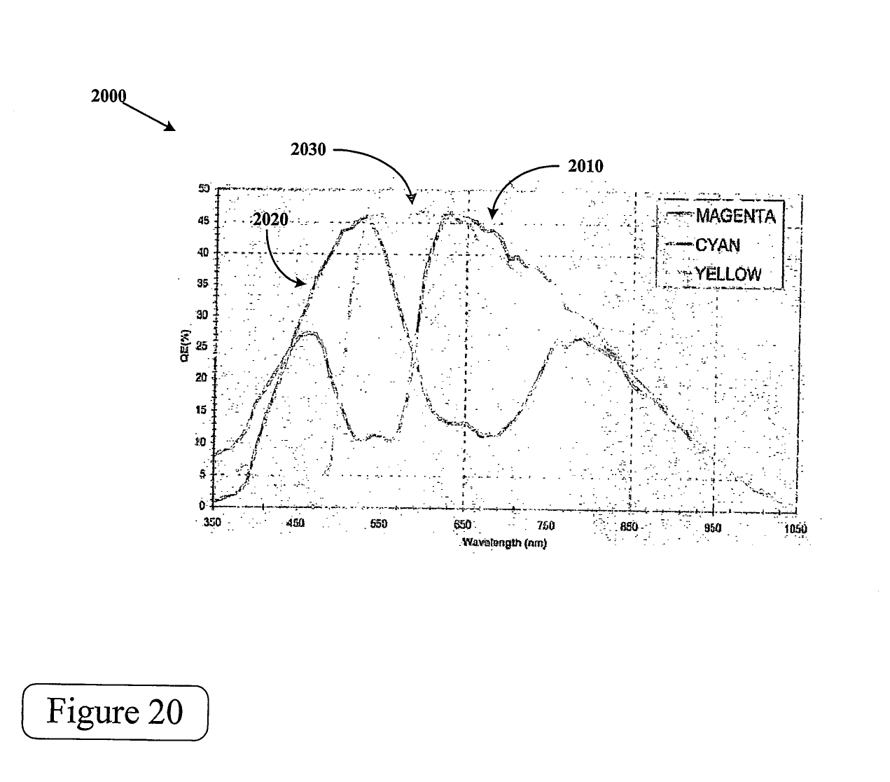

FIG. 20 illustrates a typical quantum efficiency spectral response for an image sensor. For FIG. 20, a Bayer CMY filter is evaluated. The spectral response 500 illustrates the quantum efficiency of the image sensor for various wavelengths of light incident on the image sensor. There is an individual response curve for a pixel with a magenta filter 2010, a pixel with a cyan filter 2020, and a pixel with a yellow filter 2030. Each curve has peaks and valleys at different wavelengths of incident light.

By examining the QE wavelength dependence curves for each filter type used in an image sensor, the output signal proportional to the photon flux can be determined for any wavelength or interval of interest, including those pixels for a monochromatic image sensor.

In many cases every pixel in an array is essentially identical to its neighbor except for the kind of filter (CMY, RGB, other pattern, or no filter). The effect of a filter is either to increase or to reduce the photodiode energy output for a given photon flux. The effect on the signal is wavelength dependent. The QE is the variable in the output signal that should be factored out of the equation if fair comparisons are to be made across the imaging array for each and every pixel.

In a CMOS imager, the pixel signal is obtained for each pixel as raw data after the analog to digital converter transforms the value for a set time interval. If QE is expressed as a fraction, the pixel signal is directly proportional to the product of the QE and Photon Flux:
PIXEL SIGNAL=CONSTANT×QE×PHOTON FLUX

Under an embodiment of the invention, if raw data can be normalized according to the appropriate QE, digital values can be created that may be used in subsequent analysis. The pixel value for a color CMOS image sensor is obtained before on chip conversion occurs and the value is normalized by multiplying each signal value for a particular color filter by the inverse QE.

For a relatively narrow bandwidth, the QE may be treated as a constant depending upon the wavelength and filter type used. In one example, the Bayer CMY pattern over the range 550 to 650 nm for a Kodak 1310 color CMOS image sensor provides a QE of approximately 46 percent, and then drops linearly from 650 nm to 5 percent at 990 nm, approximately 0.6 percent every 5 nm. In addition, the Magenta and Yellow filters are very similar over the range from 630 nm to 990 nm.

For a particular example with a CMY pattern Kodak 1310 color image sensor at 670 nm, the QE values are as shown in Table 9. A pixel with a yellow filter would have its digital raw data multiplied by 2.38, a magenta filter pixel by 2.27, and a cyan filter pixel by 7.69. In this embodiment, the signal for every pixel is effectively transformed to a numeric value that is directly proportional to the actual photon flux. It is noted that Table 9 only contains the QE for the image sensor when light of a particular wavelength (670 nm) strikes the image sensor. The QE for any other wavelength of light will vary, as shown in FIG. 20.

TABLE 9
Quantum Efficiency and Normalization Factors for
Kodak 1310 Image Sensor at 670 nm

| | Quantum | Normalization |
|---|---|---|
| Filter Type | Efficiency (%) | Factor |
| Yellow | 43 | 2.38 |
| Magenta | 44 | 2.27 |
| Cyan | 13 | 7.69 |
| Monochrome (no filter) | 28 | 3.57 |
| Red | 35 | 2.86 |
| Green | 5 | 20.0 |
| Blue | 3 | 33.3 |

QE Factor Correction

Corrections to account for differences in QE may be made based upon known QE factors for a particular filter type and wavelength. (For example, see the data contained in Table 8 and Table 9.) However, an image sensor may also be utilized to automatically correct for differences in pixel QE. Under an embodiment of the invention, each area of a sensor array, such as each filter quadrant, is normalized to render every pixel in the quadrant optimally tuned for photon flux in real time.

According to an embodiment, no corrections are made if the pixels and filters are all of the same type, as, for example, the YYYY, MMMM, and CCCC filter patterns shown in Table 8. A correction is made if there are two or more filter types in the array (e.g. filter patterns such as YYYC, RGB Bayer, or modified CMY Bayer). A method of auto correcting for QE can be used for any combination of two or more filter and photodiode types and such method corrects to normalize the 4 pixels in a quadrant so that each pixel produces an equivalent output signal.

If the pixels are tightly packed in a quadrant relative to the change in photon flux over a given region of the array, then it can be assumed that the same number of photons are striking each pixel in the quadrant at any given moment. With the currently available high-resolution sensors, and with anticipated future improvements in resolution, the assumption that neighboring pixels in any given quadrant experience identical photon flux is appropriate. Using this assumption, each of the pixels in the quadrant should produce the same output. According to an embodiment, auto correction causes adjacent neighbors in each filter quadrant to be identical. The most sensitive pixel type in a quadrant is used to factor out QE and wavelength differences, which simplifies the problem of correcting for wavelength and bandwidth dependence. Auto correction also reduces or eliminates problems related to temperature variations for different filter and photodiode types.

In one example, an array of an image sensor comprises multiple filter quadrants. Two or more filters are used in each quadrant of 4 pixels. In each quadrant of 4 pixels, the average analog to digital converted signal output for each filter and photodiode type is determined. If, for example, there are 3 yellow filtered pixels and 1 cyan filtered pixel in the quadrant, the average for the 3 yellow pixels is determined first. The output value for the yellow pixels is then compared to the value for the cyan pixel to determine which output is numerically greater. Under the embodiment, there is an assumption that all 4 pixels receive equivalent photon flux. The highest output value is assigned to all four pixels in the quadrant. The next quadrant in the array may then be corrected in the same manner, with the process continuing until the entire array has been assigned corrected output values to correlate with photon flux.

The process of auto correction is repeated over time as an image sensor is used to record images. In an example, the wavelength of light received by an image sensor may change from a first wavelength to a second wavelength. A first type of filter may provide the highest QE for the first wavelength, while a second type of filter provides the highest QE for the second wavelength. The change in wavelength is included in the calculation process and therefore auto correction for changing light can be made in real time.

Under an embodiment of the invention, it is not necessary to know in advance the QE for each filter type to auto correct for QE differences. Auto correcting the sensor based on the photon flux at the time an image is obtained optimizes the photo image to correlate with photon flux. This method of correcting the signal removes temperature and wavelength dependence differences for different filter types and can be implemented using software. Such method thus automatically corrects for a broad band signal impinging upon an image sensor.

Under an embodiment of the invention, the digital signals produced by an image sensor auto corrected for photon flux may be rendered to a gray scale image for subsequent visualization in a monochromatic representation.

Image Sensor Calibration

Under an embodiment of the invention, the optimization of an image sensor can first be calibrated. The calibration may be accomplished by illuminating the color filters and photodiodes with light of known wavelength and intensity. For a color CMOS imager, the raw data for each filter is obtained and compared to expected values. From the resulting comparison, the QE and the multiplier (normalization factor) that is required to obtain the equivalent output signal for each color filter used for each and every pixel in the array may be obtained.

Optimized signals obtained using QE factor conversions can more accurately relate the signal to the photon flux, and therefore more precisely characterize events, such as the optical events related to excitation-emission spectra or absorption phenomena in a chemical reaction. Both sensitivity and accuracy are enhanced by properly converting the signal to account for QE factors.

Using a standard CMOS imager (such as a Kodak 1310 color CMOS image sensor,) in an embodiment of the invention, raw data produced may be processed for signal optimization. The signal is converted to a numeric value that correlates with the photon flux incident upon the imager. This process may be applied to either a color or monochromatic CMOS imager sensor to render the signal proportional to photon flux.

Data processed according to this embodiment may be rendered for visualization, such as via a gray scale standard (0 to 255 monochromatic) to producing a black and white image that correlates with the actual photon flux. The visual image of the data is superficially equivalent to a gray scale monochromatic image sensor, but for an equivalent luminance will be more intense than a image produced by a monochromatic non-transformed CMOS counterpart because color CMOS chips are generally more sensitive than monochrome chips. A color image sensor generally provides a better signal and is more sensitive than a monochromatic imager because the pixel photodiode filters improve upon the QE for the photodiode. The filtering of light by a color image sensor may be corrected using the QE factors to convert the signal to a number that is directly proportional to the photon flux. Advantage then is taken of the color filter's microlens for every pixel, effectively amplifying the aperture for the photodiode.

Illustrations of Processes

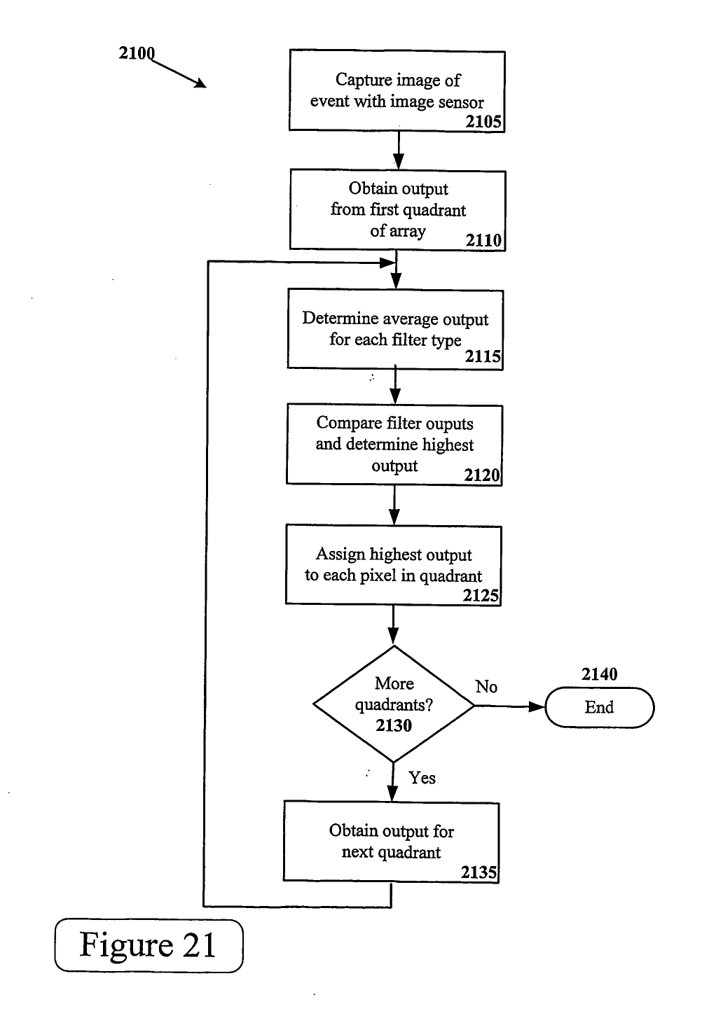

FIG. 21 is an illustration of an embodiment of correction of QE factors for an image sensor. In the correction process 2100, an image of an event is captured with an image sensor. In this illustration, the image sensor is a color sensor containing an array of pixels, with each pixel having a filter. In this case, the filters are arranged in quadrants, with each quadrant having a particular filter pattern. However, embodiments of the invention are not limited to any particular type of image sensor or filter arrangement.

In FIG. 21, the outputs of each of the pixels within a first quadrant of the array are obtained 2110. The average output of for each filter type in the quadrant is then determined. In one example, if a filter quadrant is CMY pattern containing a cyan filter, a magenta filter, and two yellow filters, the cyan output, the magenta output, and the average of the two yellow outputs are determined. The outputs are then compared and which output is highest is determined 2120. The highest output is then assigned to each pixel in the quadrant 2125. For example, if the average yellow output is the highest output for the CMY quadrant, indicating that, under the particular conditions, the yellow filter has the highest QE factor, then the average yellow output is assigned to each of the pixels in the quadrant. If there are more quadrants in the array 2130, the output of the next quadrant is obtained 2135 and the process continues. Once the final quadrant has been corrected the process is completed 2140 and the corrected output for the array is available. The process can then be repeated over time to allow real time QE factor correction for the image sensor.

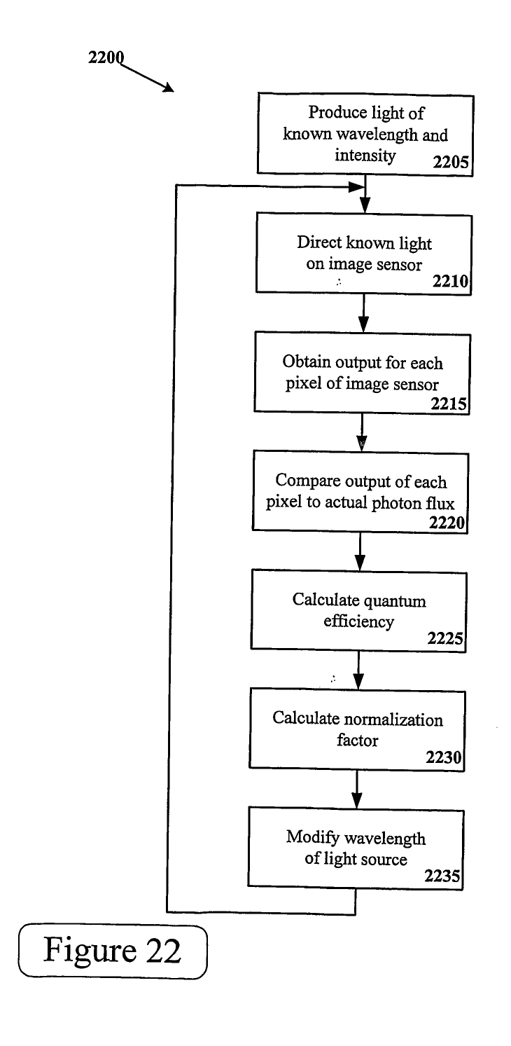

FIG. 22 is an illustration of an embodiment of a process for calibration for optimization of an image sensor. In the calibration process 2200, a light of a known wavelength and intensity is produced 2205. With a known intensity, the photon flux on each pixel is known, which would be the output if the QE of a pixel were 100 percent. The known light is directed on an image sensor 2210. The output of each pixel of the image sensor is obtained 2215. The output of the image sensor may then be compared with the actual photon flux 2220. Using the comparison, the quantum efficiency of the pixel may be calculated 2225, and then a normalization factor may be calculated based upon the quantum efficiency 2230. For a color CMOS image sensor, the comparison and calculation may be done for each filter color, resulting in a normalization factor for each filter color. In other embodiments, the comparison and calculation may be made for each pixel of an image sensor or for sectors of pixels of an image sensor, resulting in normalization factors that apply for certain portions of the image sensor. As the normalization factor varies for each wavelength of light that strikes the image sensor, the wavelength of the known light is varied 2235 and the process repeated for each needed wavelength.

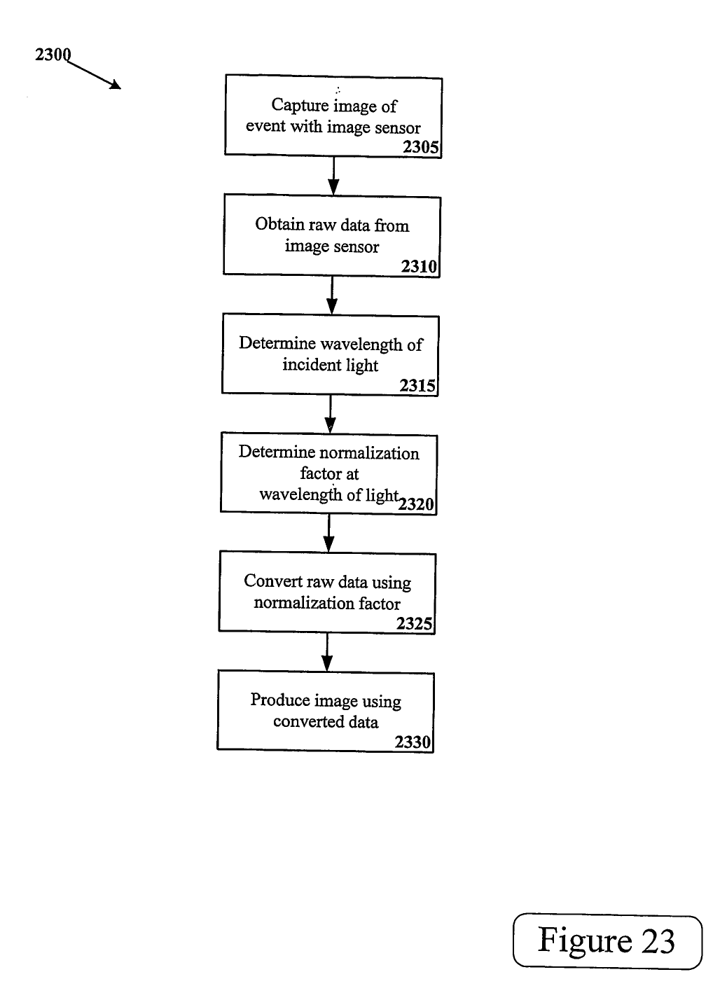

FIG. 23 is a flowchart illustrating an embodiment of the optimization of an image sensor. In the optimization process 2300, an image of an event is captured with an image sensor 2305. Under one embodiment of the invention, the image sensor is a color CMOS image sensor utilizing a filter pattern such as a Bayer RGB or CMY pattern. The raw data for the image of the event may be obtained from the image sensor 2310. The raw data is non-optimized data that, due to the nature of the image sensor, will generally vary greatly from the actual photon flux that struck the image sensor. As the normalization factor depends on the wavelength of light, the wavelength is determined 2320. The appropriate normalization factor may be determined for each pixel based upon the wavelength of light. For one embodiment utilizing a color CMOS image sensor, a normalization factor for each lens color may be used in normalization. Under other embodiments, the normalization factors may vary based on other factors. The raw data may then converted using the appropriate normalization factors for the pixels of the image sensor 2325, thus producing an optimized data set that approximates the actual photon flux for the captured image of the event. Under an embodiment of the invention, an image may be produced using the converted data 2330.

Computer Operation

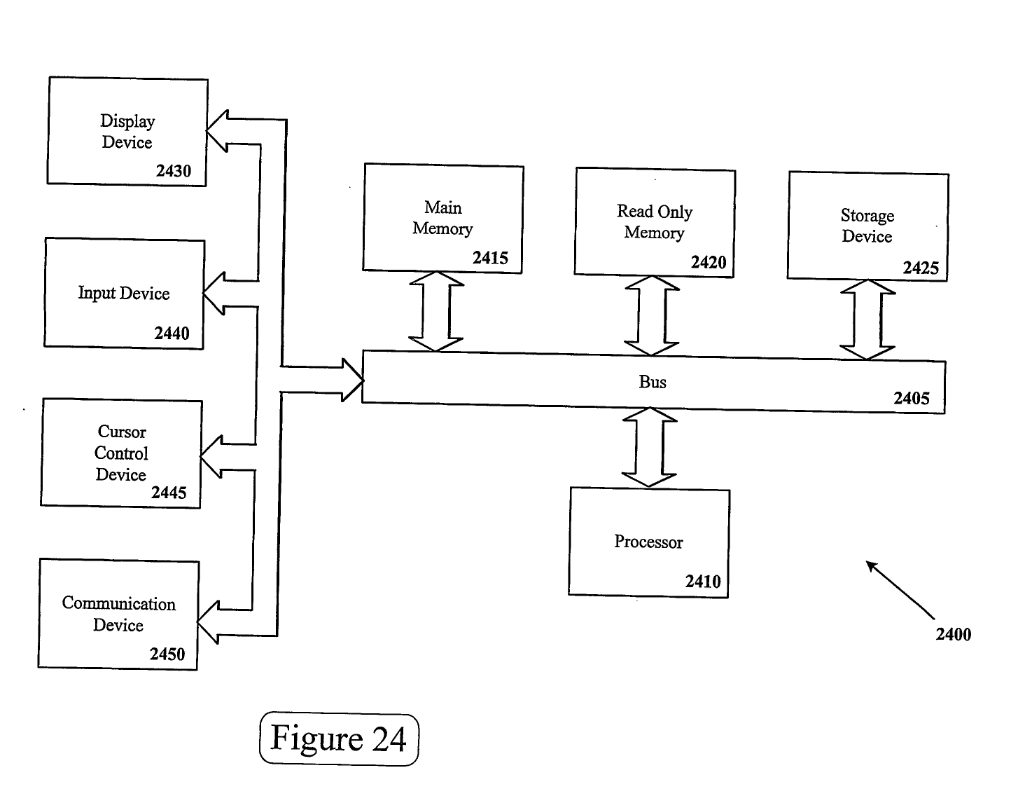

FIG. 24 is block diagram of an exemplary computer that may be used in conjunction with an image sensor in an embodiment of the invention. While FIG. 24 illustrates a computer that may be connected to the image sensor, in other embodiments the function of the components shown may be structured in varying manner or may be performed by different systems. In some embodiments some signal processing functions may be performed by the image sensor or by components coupled with the image sensor. In some embodiments an imaging system may include most or all functions in a single unit. Not all computers are structured as shown in FIG. 24. In addition, certain computers may utilize elements shown in FIG. 24 as auxiliary devices that are external from the computer.

Under an embodiment of the invention, a computer 2400 comprises a bus 2405 or other communication means for communicating information, and a processing means such as a processor 2410 coupled with the bus 2405 for processing information. The computer 2400 further comprises a random access memory (RAM) or other dynamic storage device as a main memory 2415 for storing information and instructions to be executed by the processor 2410. Main memory 2415 also may be used for storing temporary variables or other intermediate information during execution of instructions by the processor 2410. The computer 2400 also may comprise a read only memory (ROM) 2420 and/or other static storage device for storing static information and instructions for the processor 2410.

A data storage device 2425 may also be coupled to the bus 2405 of the computer 2400 for storing information and instructions. The data storage device 2425 may include a magnetic disk or optical disc and its corresponding drive, flash memory or other nonvolatile memory, or other memory device. The computer 2400 may also be coupled via the bus 2405 to a display device 2430, such as a liquid crystal display (LCD) or other display technology, for displaying information to an end user. In some environments, the display device may be a touch-screen that is also utilized as at least a part of an input device. In some environments, display device 2430 may be or may include an auditory device, such as a speaker for providing auditory information. An input device 2440 may be coupled to the bus 2405 for communicating information and/or command selections to the processor 2410. In various implementations, input device 2440 may be a keyboard, a keypad, a touch-screen and stylus, a voice-activated system, or other input device, or combinations of such devices. Another type of user input device that may be included is a cursor control device 2445, such as a mouse, a trackball, or cursor direction keys for communicating direction information and command selections to processor 2410 and for controlling cursor movement on display device 2430.

A communication device 2450 may also be coupled to the bus 2405. Depending upon the particular implementation, the communication device 2450 may include a transceiver, a wireless modem, a network interface card, or other interface device. The computer 2400 may be linked to a network or to other devices using the communication device 2450, which may include links to the Internet, a local area network, or another environment.

Fluorescence Detection

Fluorophores are frequently used to detect the presence or absence of a coupled reaction on a glass or other surface. Fluorescence detectors measure the intensity of the evanescent wave produced when a fluorophore is excited with a laser or other light source. Typically the laser is used to excite the fluorophore at its absorption peak and the detector is tuned to read the emission signal at a longer emission wavelength, which is characteristic of that particular fluorophore. The shift in wavelength between absorption and emission is referred to as the Stokes shift. Most fluorescence detection methods use fluorophores with a large Stokes shift so that the emission and absorption curves are well separated. With fluorophores that have a small Stokes shift, it is necessary to excite at a shorter wavelength than the optimal peak absorption maximum because of overlap between the emission and absorption curves. The signal emission intensity is reduced and the sensitivity for detecting target molecules is decreased. The need for a large Stokes shift also limits the choices of fluorophores that can be used.

Because the curves for absorption and emission are frequently very near to one another, accurate reading of the emission signal may be complicated. If the distance between the emission and absorption curves is small, it is difficult to separate the light from an emission spectrum from that of the absorption signal. Lasers with a narrow band at the absorption peak are frequently used with filters to cut out all light up to a certain critical point just below the emission spectral curve. By selecting an appropriate long pass filter, band pass filter, or combination of long pass and band pass filters, the emission signal can be observed in a narrow window, eliminating much of the interference from the excitatory light source. Interference from the excitatory light source is also avoided by aligning the detector and apparatus so that the emission signal can be read at a large incident angle to the excitation beam. Although filters eliminate most of the signal from the excitatory light source, they also cut out a significant portion of the evanescent (emitted) signal. Most band pass filters cut out as much as 40 to 50% of the emission signal. Long pass filters may cut an additional 10% of the emission signal.

Fluorescent detection is used in a number of common test methods. DNA hybridization is commonly analyzed in this manner, using an appropriate fluorophore coupled to a set of known oligonucleotides that hybridize to capture oligonucleotides affixed to a slide. Sandwich immunoassays also employ this method of analysis, either using a tagged secondary antibody that binds to a primary antibody, or using a secondary biotinylated antibody and an avidin-fluorophore as the tag. Many variations on this method are well known.

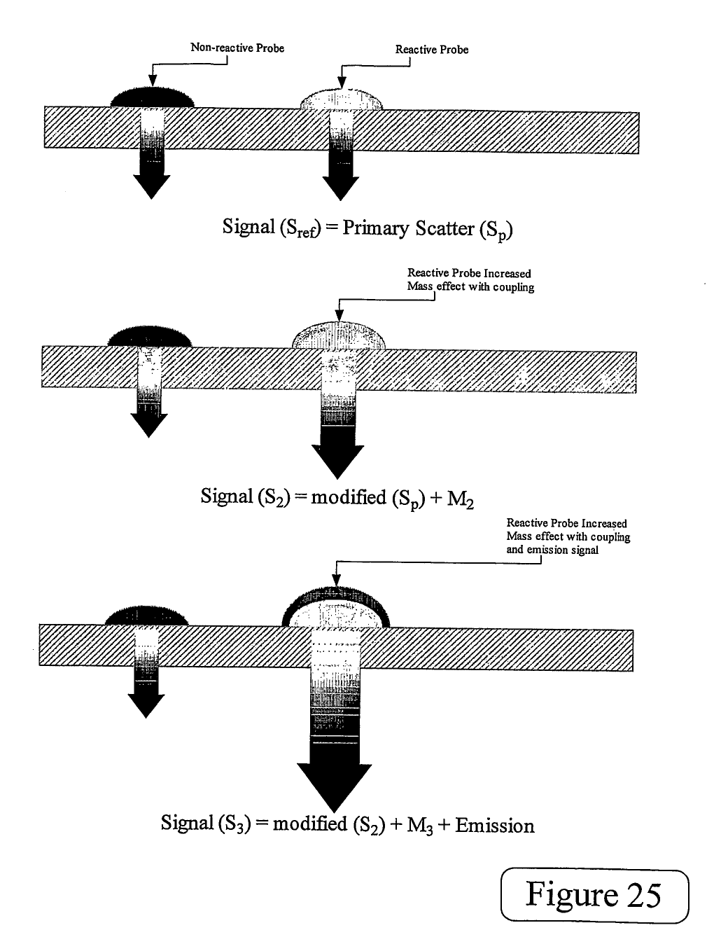

Various other types of light interference may occur in fluorescent detection. Light scatter occurs by reflection of the excitation beam, while light dispersion occurs by reflection and bending of the excitation beam. Scatter and dispersion may represent a large part of the light striking a detector. In general, when a substance (such as a protein, nucleic acid or other biomolecule) is affixed to the surface of a glass slide, it acts as a mirror to reflect and scatter light in a variety of directions. The amount of surface covered and the mass or density of the attached material may greatly affect the amount of scattered light. The chemical composition of proteins, oligonucleotides or polymers attached to the surface may also affect the scattered light (see FIG. 25). In addition, the material attached to the surface material may itself fluoresce. The glass or other material used may also have surface irregularities that can affect the signals received by the detector. The energy absorbed across the surface may vary from one spot to another, making signal analysis very problematic. Such problems require the use of novel methods of fluorescent detection and/or data analysis.

Evanescent Emission and Scattered Light

Evanescent signals are generally very weak and light scatter is intense, making accurate quantitative detection of analytes problematic. Light scatter is frequently assumed to be eliminated by filters. However, scattered light is almost always present and can be a significant part of the total signal reaching a detector. Filters used to remove light scatter also remove much of the target emission signal, thereby decreasing detector sensitivity. Filters may also transmit a small amount of scattered light. If the scattered light is relatively large compared to the evanescent emitted light, the detected signal will be a combination from several sources, only one of which represents target molecule binding.

The components of light scattering are illustrated in FIG. 25: Two spots (e.g., different antibodies) are deposited on a surface. During a method to detect a target, one of the spots remains totally non-reactive. The other spot reacts with a target, such as a bacterial pathogen and/or other reagents. Target binding to the reactive antibody increases the mass attached to the spot and results in a larger surface area and a change in molecular structure at the spot. A mass effect has occurred. The light scatter from the reactive spot will be different from the light scatter before target molecule binding. A sensitive photon-counting detector could detect this difference in scatter. A variety of instruments, such as certain flow cytometers and turbidity meters take advantage of scatter to quantify the amount of material in a solution. Those instruments measure the angle of scatter for a beam of light impinging on a target material. The change in signal is the difference between the reference signal (S_{ref}) and signal 2 (S₂). In FIG. 25 the S₂ signal is shown as having two components, a modified scatter signal plus a mass effect signal of the coupled pathogen. The signal from the reactive spot changes while the signal from the non-reactive spot signal is constant.
ΔS (non-reactive spot)=0
ΔS (reactive spot)=Modified (Sₚ)+M₁−S_{ref}

If the mass effect is sufficient to cause a large scatter effect, the fluorophore used for target detection could be eliminated. For example in DNA hybridization experiments, the mass attached to a surface using standard oligonucleotide probes (about 24 nucleotides in length) may be increased by a factor of 2 or more upon binding of target nucleic acids. Such a large change in mass may be detectable by monitoring light scatter instead of evanescent waves. In the case of a sandwich immunoassay with a biotinylated secondary antibody, another mass effect occurs when the biotinylated antibody binds to the pathogen. A third mass effect occurs when avidin-conjugated fluorophore binds to biotin.

The most sensitive signal may be obtained by subtracting the initial reference signal from the final captured signal, obtained after the fluorophore has been attached and excited. That signal represents the modified accumulated mass effects and the emission signal for the reactive spot.
ΔS (reactive spot)=Modified accumulated mass effects+Emission−S_{ref}

This method of analysis may be used with a CMOS imager or any known digital imaging method that allows storage of pixel images for subsequent processing. The signal obtained from each spot will contain more useful information and will show a more intense change upon target binding if a proper subtraction method is used. The scatter effect may be turned to an advantage in detecting target binding. Moreover, it is unnecessary to have fluorophore emission and absorption curves well separated, since spurious signals are subtracted out of the image. The full intensity of an emission signal may be measured without reducing emitted light by with filters.

A subtraction method also eliminates artifacts and defects that may derive, for example, from inhomogeneity (chips, flaws) in the slide surface. The non-reactive spots completely blank out and do not appear as a signal.

Because CMOS imagers and pixel capturing devices in general exhibit a random, very low level noise there are limits as to what kinds of signals can be detected. At any given point in time, the baseline reference may exhibit a random number of spikes. A weak signal falling between two spikes would not normally be detected against this background noise.

The signal-to-noise problem may be improved if numerous images are captured and added one upon the other. Because the random spikes inherent in a detector such as a CMOS imager are constantly shifting about, accumulating the frame images will tend to average out the random noise. However a weak signal from the emission of an excited fluorophore does not change its pixel location. Therefore, an accumulated signal caused by target binding will increase with time. This method is similar to taking a photoimage of a distant star or galaxy, by tracking the object as it moves across the sky. The object of interest appears brighter against the background with time because the signal has accumulated at the same spot on the detector, while the background light averages out.

Method of Analysis

In an exemplary embodiment of the invention, a glass slide or other matrix array is secured on a stage. A fluidic cube or other device is attached to the surface of the glass and used to deliver samples, second antibodies and other reagents. Before target molecule binding, an excitatory laser is focused on one end of the glass slide at an inclined angle about 30 to 40 degrees. The glass slide acts as a waveguide to conduct the excitatory light to spots, containing bound primary antibody, on the glass surface. A CMOS imager is used to capture the light signals. The CMOS chip is located beneath the glass slide and is aligned so that spots on the slide are directly above the imager and are sharply focused on the imager surface with optical lenses and apertures.

A number of pictures are taken. Each picture represents a single frame. For example 10 frames are taken using a 50 millisecond exposure. The exposure is selected so that the amount of light captured in a single frame is within the sensitive range, for the camera. The 10 digital frames are then added to provide a reference set that is used for subtraction of unwanted (background) signals. The accumulated image is referred to as the calibration slide.

The fluidic cube is used to expose primary antibody to a sample, bind any target molecules to the first antibody, and bind second antibody to the target. The process ends with binding of avidin-fluorophore to the biotinylated second antibody and a final set of washes. The same number of frames used to obtain the reference slide image are taken of the sample slide, using the same exposures. The cumulative set of frames is referred to as the sample slide image. The luminescent signal for each spot is determined by subtracting the reference slide image from the sample slide image. This process essentially eliminates background noise and matrix array artifacts, resulting in very sensitive detection of target molecules.

In alternative embodiments of the invention, pictures may be obtained in either still frame or video mode. A typical video frame runs at 2000 ms and captures 100 frames each for the reference and sample analysis. This method removes artifacts and non-reactive spots, leaving only those signals that represent target molecule binding to the array.

General Matters

In the description above, for the purposes of explanation, numerous specific details are set forth in order to provide a thorough understanding of the present invention. It will be apparent, however, to one skilled in the art that the present invention may be practiced without some of these specific details. In other instances, well-known structures and devices are shown in block diagram form.

The present invention may include various processes. The processes of the present invention may be performed by hardware components or may be embodied in machine-executable instructions, which may be used to cause a general-purpose or special-purpose processor or logic circuits programmed with the instructions to perform the processes. Alternatively, the processes may be performed by a combination of hardware and software.

Portions of the present invention may be provided as a computer program product, which may include a machine-readable medium having stored thereon instructions, which may be used to program a computer (or other electronic devices) to perform a process according to the present invention. The machine-readable medium may include, but is not limited to, floppy diskettes, optical disks, CD-ROMs, and magneto-optical disks, ROMs, RAMs, EPROMs, EEPROMs, magnet or optical cards, flash memory, or other type of media/machine-readable medium suitable for storing electronic instructions. Moreover, the present invention may also be downloaded as a computer program product, wherein the program may be transferred from a remote computer to a requesting computer by way of data signals embodied in a carrier wave or other propagation medium via a communication link (e.g., a modem or network connection).

Many of the methods are described in their most basic form, but processes can be added to or deleted from any of the methods and information can be added or subtracted from any of the described messages without departing from the basic scope of the present invention. It will be apparent to those skilled in the art that many further modifications and adaptations can be made. The particular embodiments are not provided to limit the invention but to illustrate it. The scope of the present invention is not to be determined by the specific examples provided above but only by the claims below.

It should also be appreciated that reference throughout this specification to “one embodiment” or “an embodiment” means that a particular feature may be included in the practice of the invention. Similarly, it should be appreciated that in the foregoing description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims are hereby expressly incorporated into this description, with each claim standing on its own as a separate embodiment of this invention.

### EXAMPLES

### Example 1

### Microarray Formation Using Translucent Nitrocellulose Coated Slides

Colloidal nitrocellulose slides may be spotted with probes using any known methods for microarray production. Methods for spotting proteins, peptides, oligonucleotides and nucleic acids onto nitrocellulose surfaces are well known in the art. Antibodies and biotinylated bovine serum albumin were used to determine the colloidal nitrocellulose binding capacity. The estimated protein binding capacity for the initial matrix arrays was in the range of 100 to 200 μg/cm².

A CMOS imaging system that detected light emissions through the translucent nitrocellulose matrix arrays was used with a Cy5-streptavidin indicator dye. (See, e.g., U.S. patent application Ser. No. 09/974,089, filed Oct. 1, 2001.) At a loading volume of approximately 5 nanoliters per spot, biotinylated BSA was reproducibly detected at least down to 10 to 20 picograms of protein. Optimal CMOS images were obtained using a protein concentration range of about 20 to 100 μg/mL biotinylated BSA. The CMOS imaging data was confirmed using a 24 hour colloidal gold stain BioRad, Hercules, Calif.) of the spot arrayed translucent nitrocellulose slides.

The ability of the colloidal nitrocellulose matrix to maintain antibody binding activity was confirmed by spotting a variety of antibodies on the translucent surface. A concentration range of 20 to 200 μg/mL was used. Nonspecific protein binding sites on the nitrocellulose membrane were blocked with 0.1% BSA in buffer solution. The antibodies were then exposed to solutions containing the appropriate target antigen. Binding was detected using biotinylated second antibodies and Cy5-streptavidin indicator.

A 2D array spotted with 5 nanoliters per spot of primary goat anti-mouse antibody solution and was developed using a secondary mouse Listeria monoclonal antibody and a secondary biotinylated goat anti-mouse antibody. In this assay, the primary and secondary goat anti-mouse antibodies bound to different epitopes of the secondary mouse antibody. The two secondary antibodies were thus used to detect primary antibody bound to the array. Using a CMOS imaging system, the primary antibody could be detected down to a level of 100 picograms or less of antibody. The CMOS data was confirmed on the same slides using colloidal gold staining.

### Example 2

### Bioluminescent Detection Sensitivity for Virus Analytes

Rous sarcoma virus (RSV) was titered using primary antibody probe binding and HRP-SA bioluminescent detection as disclosed above. Prespotted slides containing anti-RSV antibodies were prepared for RSV. RSV was serially diluted 10 fold with each dilution to finally reach a lower limit of detection where no further spots were detected. A lower limit of detection was estimated at is 0.000091 IU/mL for bioluminescent detection (10¹³ fold dilution from the initial stock titer).

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE 1
Pre-coated Blank Footpad Procedure

| | Time |
|---|---|
| | (mins) |
| Remove foil seal exposing first the footpad in the A reservoir. | |
| Carefully remove the footpad with an inverted eppendorf. | |
| Apply with an eppendorf a microspot index mark at the 12 o′clock | 1 |
| position using an index marker. | |
| Apply from 1 to approximately 8 additional microspots on the footpad | 2 |
| in a pattern noting and recording accurately the position of each spot | |
| relative to the index. | |
| Allow approximately 30 minutes air drying in a clean protected area. | 3 |
| Prepare secondary biotinylated antibodies diluting with dilution buffers | |
| to an appropriate level (approximately 10-30 uG/mL). Do not use | |
| sodium azide or other antimicrobial growth inhibitors because they will | |
| interfere in the test. Dilution buffers are formulated with the | |
| appropriate inhibitors.¹ | |
| Prepare the test sample that will be added to reservoir A. | |
| Prepare luminol-peroxide solutions mixing equal volumes of PriTest | 1 |
| provided substrate. | |
| Remove the foil covering reservoirs B through H. | |
| Add 100 uL luminol-peroxide substrate (step 8) to reservoir 8. | |
| Add 200 uL biotinylated antibody (step 6) to reservoir D. | |
| Place footpad face down into reservoir B (blocking solution) and allow | 5 |
| 5 minutes incubation. | |
| Place test sample (step 7) in reservoir A. | |
| Place footpad face down into reservoir A, and allow 10 minutes | 10 |
| incubation. | |
| Remove footpad from A reservoir, immerse briefly in B to wash the | 10 |
| surface. Then move directly to reservoir C and briefly immerse and | |
| wash the surface, then place the footpad in D to incubate 10 minutes. | |
| Remove the footpad from D reservoir, wash in E briefly, and place it in | 10 |
| reservoir F to incubate 10 minutes. | |
| Remove the footpad from F reservoir, wash in G briefly, and place it in reservoir H. | |
| Immediately place the microplate on the TOAD stage and begin | |
| imaging. | |
| Imaging may take 15 minutes for very low level detection. | 15 |

¹ProClin 0.05%.

TABLE 2
Pre-Arrayed Footpad Procedure

| | Time |
|---|---|
| | (mins) |
| Remove foil seal exposing first the footpad in the A reservoir. | |
| Carefully remove the footpad with an inverted eppendorf from reservoir A and place it | 5 |
| face down in reservoir B (blocking solution) and allow 5 minutes incubation | |
| Prepare the test sample that will be added to reservoir A. | |
| Prepare luminol-peroxide solutions mixing equal volumes of PriTest provided | 1 |
| substrate. | |
| Remove the foil covering reservoirs C through H. | |
| Add 100 uL luminol-peroxide substrate (step 4) to reservoir 8. | |
| Place test sample (step 3) in reservoir A. | |
| Remove the footpad from reservoir B and place the footpad face down into reservoir | 10 |
| Allow 10 minutes incubation. | |
| Remove footpad from A reservoir, immerse briefly in B to wash the surface. Then | 10 |
| move directly to reservoir C and briefly immerse and wash the surface, then place the | |
| footpad in D to incubate 10 minutes. | |
| Remove the footpad from D reservoir, wash in E briefly, and place it in reservoir F to | 10 |
| incubate 10 minutes. | |
| Remove the footpad from F reservoir, wash in G briefly, and place it in reservoir H. | |
| Immediately place the microplate on the TOAD stage and begin imaging. | |
| Imaging may take 15 minutes for very low level detection. | 15 |

TABLE 3
Component Configuration:

| | | Units per 96 Well | |
|---|---|---|---|
| | | Microplate | |
| Item | Composition | (per well/total) | Well |
| Footpad | E.g. rayon, nylon, | 1/12 | A |
| | cellophane | | |
| Spotting buffer | PBS (phosphate buffered | N/A | |
| | saline) | | |
| Dilution buffer | 0.1% BSA, PBS | N/A | |
| | 0.05% ProClin 950 | | |
| Index buffer | PBS, 0.05% ProClin 950 | N/A | |
| | 30 uG/mL B-BSA | | |
| Blocking solution | PBS, 0.1% BSA | 100 uL/1200 uL | B |
| | 0.05% ProClin 950 | | |
| Wash solution | PBS | 100 uL/3600 uL | C |
| | 0.1% BSA | | E |
| | 0.05% ProClin 950 | | G |
| | 0.03% Triton X100 | | |
| HRP-SA | PBS, 0.1% BSA | 100 uL/1200 uL | |
| | 0.05% ProClin 950 | | F |
| | 0.03% Triton X100 | | |
| | 0.1 uG/mL HRP-SA | | |
| Luminol-peroxide | Mix 1:1 volumes prior to | 100 uL/1200 uL | H |
| | test procedure and fill H | | |
| | reservoir | | |
| Antibody Cocktail | PBS | | |
| | 0.1% BSA | | |
| | 0.05% ProClin 950 | 100 uL/1200 uL | D |
| | 0.03% Triton X100 | | |
| | Biotinylated Antibodies | | |

TABLE 4
Pre-spotted Array Procedure

| | Time (min) |
|---|---|
| Prepare the test sample that will be used. The sample may be immobilized in | |
| dilution buffer. | |
| Prepare luminol-peroxide solutions mixing equal volumes of provided substrate. | 1 |
| Dilute the mixed reagents with a volume of dilution buffer equal to the total | |
| luminol + peroxide (example: 1 mL Luminol + 1 mL peroxide, then dilute with 2 mL | |
| dilution buffer to produce 4 mL total test reagent). | |
| Block the prepared slide by filling the incubation sleeve chamber with blocking | 10 |
| solution and allowing 10 minutes for complete blocking. Then remove and | |
| dispose of blocking solution. | |
| Place test sample into the incubation chamber and allow 10 minutes for probe | 10 |
| binding to occur. Then empty the reservoir contents and dispose of remaining | |
| sample. | |
| Wash × 1 the incubation chamber with wash solution by completely filling and | 1 |
| then emptying the reservoir chamber. | |
| Fill the reservoir chamber with the secondary biotinylated antibody, and incubate | 10 |
| 10 minutes. Then remove and dispose of the fluid. | |
| Wash × 1 the incubation chamber with wash solution by completely filling and | 1 |
| then emptying the reservoir chamber. | |
| Fill the chamber with HRP-SA reagent and incubate 10 minutes. Then empty and | 10 |
| dispose the spent reagent. | |
| Wash twice (×2) the incubation chamber with wash solution by completely | 2 |
| filling and then emptying the reservoir chamber. | |
| Fill the reservoir chamber with Luminol-peroxide reagent. Peel away the outer | |
| protective surface on the incubation sleeve, exposing the optically clear window. | |
| Place the slide on the TOAD stage. Position the slide to accurately view the target | 1 |
| area. This may be confirmed using the Grid-template slide and centering the | |
| diamond as needed. | |
| Open the software Slider2, and set the exposure time at 10 minutes | |
| Set the Readout speed at 2.5 Mhz. | 2 |
| Click “Capture Image”. | 1 |
| After the image is obtained, make notes and save the file. The ID code on the slide | 1 |
| may be used as the file name. | |

TABLE 5
Pre-coated Blank Slide Procedure

| | Time |
|---|---|
| | (min) |
| With gloves, place a slide on the spotting template with the logo face up. The | |
| target area for probe spotting is between the diamond points. Mark a slide with | |
| a label using a black marker pen. | |
| Apply with an eppendorf pipette tip, a microspot index mark at one end of the | 1 |
| target area spacing spots in a column. Note the position in a notebook, making a | |
| sketch of the slide. | |
| Apply from 1 to 8 additional columns of microspots on the slide in a pattern, | 2 |
| noting and recording accurately the position of each probe spot relative to the | |
| index in a notebook. Space columns at least 1 mm apart | |
| Allow approximately 30 minutes air drying in a clean protected area. | 30 |
| Prepare secondary biotinylated antibodies diluting with dilution buffers to an | |
| appropriate level (approximately 10-30 uG/mL). Do not use sodium azide or | |
| other antimicrobial growth inhibitors because they will interfere in the test. | |
| Dilution buffers are formulated with the appropriate microbial inhibitors.¹ | |
| Affix the incubation sleeve to the slide, encompassing the spots deposited and | 1 |
| air-dried. | |
| Prepare the test sample that will be used. The sample may be immobilized in | |
| dilution buffer. | |
| Prepare luminol-peroxide solutions, mixing equal volumes of provided | 1 |
| substrate. Dilute the mixed reagents with a volume of dilution buffer equal to | |
| the total luminol + peroxide (example: 1 ml Luminol + 1 mL peroxide, dilute | |
| with 2 mL dilution buffer to produce 4 mL total test reagent). | |
| Block the prepared slide by filling the incubation sleeve chamber with blocking | 10 |
| solution and allowing 10 minutes for complete blocking. Then remove and | |
| dispose of in a waste container. | |
| Place test sample into the incubation chamber and allow 10 minutes for probe | 10 |
| binding. Then empty the reservoir contents and dispose of in a waste container. | |
| Wash × 1 the incubation chamber with wash solution by completely filling and | 1 |
| then emptying the reservoir chamber. | |
| Fill the reservoir chamber with the secondary biotinylated antibody, and | 10 |
| incubate 10 minutes. Then remove the fluid and dispose of in a waste container. | |
| Wash × 1 the incubation chamber with wash solution by completely filling and | 1 |
| then emptying the reservoir chamber. | |
| Fill the chamber with HRP-SA reagent and incubate 10 minutes. Then empty | 10 |
| and dispose of the spent reagent in a waste container. | |
| Wash twice (×2) the incubation chamber with wash solution by completely | 2 |
| filling and then emptying the reservoir chamber. | |
| Fill the reservoir chamber with Luminol-peroxide reagent. Peel away the outer | 1 |
| protective surface on the incubation sleeve exposing the optically clear window | |
| and then place the slide on the TOAD stage. Position the slide to accurately | |
| view the target area. This may be confirmed using the Grid-template slide | |
| centering the diamond as needed. | |
| Open the software Slider2, and set the exposure time at 10 minutes | 2 |
| Set the Readout speed at 2.5 Mhz. | 1 |
| Click “Capture Image” | 1 |
| After the image is obtained, make notes and save the file. The ID code on the | |
| slide may be used as the file name. | |

¹ProClin 0.05%.

TABLE 6

| Part # | Description | Components | Supplier | PriTest Part # | Ref # | Quantity |
|---|---|---|---|---|---|---|
| Instrument: | Toad ™ Illuminator | | | FD-TI0001-0-001 | | |
| | Toad ™ Illuminator | CCD | Qimaging | | QIC-M-10-C | 1 |
| | | PCI firewire card | Qimaging | | | 1 |
| | | Lens | Sunex | | DSL914D2 | 1 |
| | | Lens C-mount adapter | Paul Harris FD | | | 1 |
| | | Firewire 6 pin | Qimaging | | | 1 |
| | | Emerge Enclosure | Emerge | | | 1 |
| | | Software interface | Validio | | | 1 |
| | | Cooper Cooler-Heat | Thermaltake | | A1349 | 1 |
| | | Sink: GF4Ti Cooper | | | | |
| | | Cooler | | | | |
| | | Philmore DC Power | Fry's | | | |
| | | Jack 2.1 mm × 5.5 mm | Electronics | | | 1 |
| | | CA-979/500 mA AC | | | | |
| | | Universal PWR adaptor | Fry's | | | |
| | | UL | Electronics | | | 1 |
| | | Shipping carton | | | | 1 |
| | | Label | | | | 1 |

TABLE 7

| | Grid Focus Slide | | PriTest | | | 1 |
|---|---|---|---|---|---|---|
| | Spotting Template | | PriTest | FD-FS0006-0-001 | | 1 |
| Part # | Description | Components | Supplier | FD-ST0005-0-001 | Ref # | Quantity |
| Disposables: | Grabber ™ | Nylon 75 mm × 25 mm × 1 n | Emerge | | | 1 |
| | | Nitrocellulose | | | | |
| | | Configuration 1 | | FD-GR0003-0-001 | | 25 |
| | | Configuration 2 | | | | 100 |
| | Incubation sleeves | 500 uL adherent chambe | Gasket Sp | | Chamber BIC | 1 |
| | | Configuration 1 | | FD-IS0004-0-003 | Chamber BIC | 25 |
| | | Configuration 2 | | | Chamber BIC | 100 |
| | Blocking Solution (mL) | BSA 0.1% w/v | Sigma | | A3803? | |
| | | ProClin 950 0.05% w/v | Supelco | | 46878-U | |
| | | Sigma PBS pack | Sigma | | P3813 | |
| | | Configuration 1 | | SK-BS1001-0-001 | | 100 |
| | | Configuration 2 | | | | 1000 |
| | Dilution Buffer (mL) | BSA 0.1% w/v | Sigma | | A3803 | |
| | | ProClin 950 0.05% w/v | Supelco | | 46878-U | |
| | | Sigma PBS pack | Sigma | | P3813 | |
| | | Configuration 1 | | SK-DB1002-0-001 | | 500 |
| | | Configuration 2 | | | | 1000 |
| | Wash Solution (mL) | BSA 0.1% w/v | Sigma | | A3803 | |
| | | ProClin 950 0.05% w/v | Supelco | | 46878-U | |
| | | Sigma PBS pack | Sigma | | P3813 | |
| | | Configuration 1 | | SK-WS1003-0-001 | | 100 |
| | | Configuration 2 | | | | 500 |
| | Spotting Buffer (mL) | Sigma PBS pack | Sigma | | | |
| | | Configuration 1 | | SK-SB1004-0-001 | | 25 |

1-18. (canceled)

19. A method of data analysis comprising:
capturing a plurality of calibration images using an imager, the image comprising a plurality of pixels;
obtaining a plurality of pixel signals for each of the plurality of calibration images;
creating an average interpolation function to produce interpolated average signal values for the imager; and
creating an interpolation function for each pixel to produce interpolated signal values for the pixel.

20. The method of claim 19, wherein each of the plurality of calibration images is at a different exposure level.

21. The method of claim 19, wherein the exposure levels for the calibration images are spaced evenly over a range of exposure levels.

22. The method of claim 19, wherein the average interpolation function and the interpolation function for each pixel are based on linear interpolation.

23. The method of claim 19, further comprising:
capturing a test image at a test exposure level;
obtaining a plurality of pixel signals for the test image; and
producing a plurality of corrected pixel signal values for the test image.

24. The method of claim 23, wherein producing the plurality of corrected signal values for the test image comprises multiplying each of the plurality of pixel signals by the average interpolation function for the imager divided by the interpolation function for the pixel.

25. The method of claim 23, further comprising producing a plurality of leveled signal values for the plurality of corrected signal values.

26. The method of claim 25, wherein producing the plurality of leveled signal values for the plurality of corrected signal values comprises:
producing a histogram of the plurality of corrected signal values;
determining a high threshold and a low threshold for the histogram; and
determining leveled signal values based on comparisons between the corrected test signals and the high threshold and low threshold.

27. The method of claim 26, further comprising setting a leveled signal value for a pixel to zero if a corrected test signal value for the pixel is less than the low threshold.

28. A machine-readable medium having stored thereon data representing sequences of instructions that, when executed by a processor, cause the processor to perform operations comprising:
receiving a plurality of pixel signal values for each of a plurality of calibration images captured by an imager;
creating an average interpolation function to produce interpolated average signal values for the imager; and
creating an interpolation function for each pixel to produce interpolated signal values for the pixel.

29. The medium of claim 28, further comprising instructions that, when executed by the processor, cause the processor to perform operations comprising:
producing leveled signal values for the plurality of corrected signal values.

30. The medium of claim 29, wherein producing leveled signal values for the plurality of corrected signal values comprises:
producing a histogram of the corrected signal values;
determining a high threshold and a low threshold for the histogram; and
determining leveled signal values based on comparisons between the corrected test signals and the high threshold and low threshold.

31. The medium of claim 30, further comprising instructions that, when executed by the processor, cause the processor to perform operations comprising:
setting a leveled signal value for a pixel to a depth value for the imager multiplied times a difference between the corrected test signal for the pixel and the low threshold and divided by a difference between the high threshold and the low threshold if the corrected signal value for the pixel is greater than or equal to the low threshold and less than or equal to the high threshold.

32. A total optical assay device comprising:
a light-tight casing with a lid;
a stage below the lid, the stage to hold slides or microtiter well plates;
a focusing lens below the stage;
an imaging device below the lens, the imaging device arranged to obtain optical images of slides or microtiter well plates on the stage;
a base to hold the imaging device in position; and
a cooling device.

33. The device of claim 32, further comprising a machine readable medium according to claim 28.

34. An optical assay device comprising:
a circular disk to fit into a well of a microtiter plate;
a probe-binding membrane attached to the bottom of the disk;
a stem attached to the top of the disk; and
a handle attached to the top of the stem.

35. The device of claim 34, wherein the membrane is rayon, cellophane or translucent nitrocellulose.

36. The device of claim 34, further comprising a multiplicity of spots bound to the lower surface of the membrane.

37. (canceled)
